(19) United States
(12) Patent Application Publication
(10) Pub. No.: US 2020/0320645 A1
(43) Pub. Date: Oct. 8, 2020

(54) SYSTEMS AND METHODS FOR FILTERING SOCIAL MEDIA INTERACTIONS AND ONLINE CONTENT BASED ON PERSONAL GENETIC PROFILES

(71) Applicant: Orig3n, Inc., Boston, MA (US)

(72) Inventors: Robin Y. Smith, Boston, MA (US); Marcie A. Glicksman, Boston, MA (US); Shadrack Cgar Frazier, Boston, MA (US)

(21) Appl. No.: 16/904,972

(22) Filed: Jun. 18, 2020

Related U.S. Application Data
(63) Continuation of application No. PCT/US18/35259, filed on May 31, 2018.
(60) Provisional application No. 62/607,779, filed on Dec. 19, 2017.

(51) Int. Cl. G06Q 50/00; G16B 20/00; G16B 50/00; G06F 16/335; G06F 16/9035; H04W 4/02; G06F 3/048

(52) U.S. Cl. CPC G06Q 50/01; G16B 20/00; G16B 50/00; G06F 3/048; G06F 16/9035; H04W 4/023; G06F 16/337

(57) ABSTRACT

Presented herein are systems and methods for automatically managing and filtering social media interactions and/or online content based on genetic profiles of users. These genetic profiles include identifications of genetic interests of the users and/or genetic information to which they have permitted access. Such permissioned personal genetic profiles may be used to facilitate, manage, and filter a variety of online interactions between the user and other members of various services and/or online content in order to lead to meaningful and successful real world interactions and/or to improve the relevance of online content delivered to a user.

### CROSS REFERENCE TO RELATED APPLICATIONS

This application claims priority to and benefit of U.S. Provisional Application No. 62/607,779, filed Dec. 19, 2017, the content of which is hereby incorporated by reference in its entirety.

### FIELD OF THE INVENTION

This invention relates generally to systems and methods for automatically managing and filtering social media interactions and/or online content for users of personal genetic profiles.

### BACKGROUND OF THE INVENTION

Genomes hold valuable information that can be used to better understand human biological characteristics and traits. Much research is being conducted to establish relationships between the human genome and biological characteristics and traits, in particular. For example, single nucleotide polymorphisms (SNPs) are specific sites identified in particular genes that influence biological characteristics and traits depending on the particular polymorphism of an individual. Different polymorphisms of the nucleotides at a specific site influence the relevant characteristic or trait differently. Relationships between the variants of SNPs and their corresponding biological characteristics and traits have been established and many more possible relationships are currently undiscovered and under investigation.

Personalized genetic profiles, such as LifeProfile™ offered by Orig3n, Inc. of Boston, Mass., provide SNP-based assessments of various characteristics and traits using simple cheek swab samples, providing secure, user-friendly, smartphone accessible test results. Individuals provide a biological sample and receive an assessment of their genetic profile that is accessible for review on their smartphones. Individuals can learn how their genome impacts their personal health characteristics, fitness characteristics, dietary characteristics, and even aspects of their personality, such as their response to stress and level of risk aversion or risk seeking behavior.

In order to act on the insight into their genetic makeup that an individual obtains through a personal genetic profile assessment, the individual may seek the advice of others, in person, as well as through online forums such as social media platforms and message boards. Without any way to target other individuals and/or content that may be especially relevant, this approach can be daunting, haphazard, and inefficient.

Accordingly, there exists a need for systems and methods to assist individuals in making meaningful and relevant connections with others, both in the real-world and via online forums.

### SUMMARY

Presented herein are systems and methods for automatically managing and filtering social media interactions and/or online content based on genetic profiles of users. These genetic profiles include identifications of genetic interests of the users and/or genetic information to which they have permitted access. Such permissioned personal genetic profiles may be used to facilitate, manage, and filter a variety of online interactions between the user and other members of various services and/or online content in order to lead to meaningful and successful real world interactions and/or to improve the relevance of online content delivered to a user.

For example, in certain embodiments, the systems and methods described herein leverage permissioned personal genetic profiles to facilitate identifying, presenting, and matching a user to members of a dating pool in order to identify members of a dating service with whom they may be compatible. In particular, the user's permissioned personal genetic profile may be compared with those of other members of the dating pool, and potential matches identified, based at least in part on the comparison between the user's permissioned personal genetic profile and that of the other members. Notably, identifying potential matches in this manner does not correspond to merely identifying genetically similar individuals, but can include taking into account desirable dissimilarities in genetic information between the user and their potential matches. For example, an individual with a genetic predisposition towards risk aversion may benefit from, and enjoy a relationship with another who has a genetic predisposition to risk seeking behavior, and vice versa.

In certain embodiments, the systems and methods described herein utilized a combination of permissioned personal genetic profiles and location data, such as GPS coordinates to allow a user to identify nearby genetic profile matches. Identifying nearby genetic profile matches can be helpful, for example, if various individuals would benefit from coordinating their activities with genetically similar individuals and/or individuals with complementary genetic traits. For example, around lunchtime, members of a genetic profile matching service may benefit from identifying other members with similar dietary needs, restrictions, and even tastes. An individual with a dietary restriction (e.g., an allergy) at an unfamiliar cafeteria may benefit from identifying another individual with the same restriction, and asking them what they ordered. A group of individuals with genetic predisposition to high cholesterol and fat storage may join together and seek out heart healthy food as a team, so as to increase their enjoyment of and adherence to a healthy diet. In workout classes or on group runs, members with similar physical fitness characteristics may benefit from sticking together, for example to pace themselves appropriately and avoid injury.

The systems and methods described herein also provide for the automated identification and filtering of online content in a genetically tailored fashion. In particular, in certain embodiments, members of online forums, including message boards and social media feeds, each provide a permissioned personal genetic profile that can be accessed by software responsible for ingesting content from various users (e.g., such as social media posts, message board posts, and the like) and delivering it to others (e.g., in the form of social medial feeds, message board threads, and the like). Accordingly, a user can be matched to various content objects—postings, message threads, and the like—based on their permissioned personal genetic profile and those of the users that created the content objects.

Content that is not necessarily created by individual users, but by, for example, various companies and media outlets, can also be delivered to users in a genetically tailored fashion. In particular, content that is not associated with a particular individual user's permissioned personal genetic profile can be associated with a content-specific genetic profile that identifies genetic interests and/or a subset of user genotypes for which the content is relevant. Such content-specific genetic profiles can be created by developers who create the content as well as automatically by, for example a news aggregator service. This approach may be particularly valuable for companies who wish to advertise particular products within online forums via sponsored content on social media feeds in a genetically targeted fashion.

As described herein, in certain embodiments, an individual's permissioned personal genetic profile uses one or more accessible genetic profile objects to represent the particular genetic interests of a given individual and/or the portion of their genetic information to which they have granted access. Genetic profile objects may include genetic interest identifiers that identify particular SNPs, genes, and groups thereof of interest to the user, as well as variant objects that represent particular variants of particular SNPs that the user has (e.g., as determined via genotyping measurements).

Accessible genetic profile objects may also include secondary profile tags that represent particular physical and/or behavioral traits derived from genotyping data for the user, but not necessarily directly corresponding to a single trait influenced by a single SNP and/or gene variant. For example, a set of dietary tags may be derived from genotyping data for the user, and used to represent particular diets to which the user should conform and/or allergens that they should avoid as part of a dietary profile for the individual. Another example of secondary profile tags includes user-specific fitness tags that identify specific workout classifications (e.g., that are recommended for the user; e.g., that the user should avoid) (e.g., alphanumeric strings such as “HIIT”, “aerobic”; “cardio”; “high intensity”, “flexibility”, and the like) having been determined as associated with (e.g., beneficial to) the user based on their genotyping data. Such secondary profile tags not only generalize genotyping data in a useful fashion, but also offer a layer of obfuscation between an individual's permissioned personal genetic profile and their specific genotyping data.

Moreover, a user may populate, review, and update their permissioned personal genetic profile in accordance with their level comfort in sharing their genetic information and/or information derived therefrom.

Accordingly, the systems and methods described herein provide for useful managing and filtering of a variety social media interactions and/or online content in a genetically tailored fashion. The approaches described herein facilitate the making of meaningful and useful real life connections between members of various genetic matching services, as well as the delivery of customized genetically tailored online forum posts and content to users.

In one aspect, the invention is directed to a method of automatically identifying, presenting, and matching a user to members of a dating pool based on genetic interests and/or shared genetic information via a graphical user interface (GUI), the method comprising: (a) accessing, by a processor of a computing device, a permissioned personal genetic profile of the user that identifies genetic interests of the user (e.g., preferred genetic traits of a potential match for the user, and/or preferred categories of traits with which to compare the user with other members of the data pool for identification of a potential match for the user) and/or genetic information to which the user has permitted access by the processor; (b) accessing, by the processor, permissioned personal genetic profiles of each of one or more of the members of the dating pool; (c) for each of a subset of the one or more members of the dating pool, determining, by the processor, the member to be a potential match based at least in part on the permissioned personal genetic profile of the user and the permissioned personal genetic profile of the member, thereby automatically identifying one or more potential matches for the user; and (d) causing, by the processor, graphical rendering of, for presentation to the user within the GUI (e.g., for presentation on a user's mobile computing device), graphics and/or text corresponding to at least a portion of a biographical profile of each of the one or more potential matches.

In certain embodiments, at least one of the potential matches is linked to a first interest indicator corresponding to the user and having an affirmative value, and the method comprises: (d) receiving, by the processor, via the GUI, a user input corresponding to second interest indicator corresponding to the at least one potential match and having an affirmative value; (e) determining, by the processor, the at least one potential match to be a positive match for the user based on both the first and second interest indicators having affirmative values; and (f) responsive to the determining the at least one potential match to be a positive match for the user, issuing, by the processor, a notification to the user of the positive match.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in a potential match with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, for each of one or more members (e.g., up to all) of the dating pool, the permissioned personal genetic profile of the member comprises one or more genetic interest identifiers (e.g., textual labels) for the member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of one or more members (e.g., up to all) of the dating pool, the permissioned personal genetic profile of the member comprises one or more accessible variant objects for the member, each representing a particular variant of a particular SNP that the member has (e.g., as determined via genotyping measurements) (e.g., each accessible variant object comprising a measurement outcome that identifies the particular variant that the member has and/or a qualifier that classifies the particular variant that the member has), and to which the member has granted access.

In certain embodiments, for each of one or more members (e.g., up to all) of the dating pool, the permissioned personal genetic profile of the member comprises one or more secondary profile tags (e.g., dietary profile tags; e.g., physical fitness tags) for the member, the secondary profile tags having been determined based on genotyping data for the member.

In certain embodiments, for each of one or more members (e.g., up to all) of the dating pool, the permissioned personal genetic profile of the member comprises one or more member-specified weighting values, each associated with a specific accessible genetic profile object (e.g., a genetic interest indicator, accessible variant object, or secondary profile tag) of the permissioned personal genetic profile of the member and indicating a level of desired similarity and/or dissimilarity and/or compatibility in a potential match with respect to the specific accessible genetic profile object of the permissioned personal genetic profile of the member (e.g., the genetic interest indicator, accessible variant object, or secondary profile tag).

In certain embodiments, step (c) comprises, for each of one or more members of the dating pool: computing a compatibility index for the member and the user based at least in part on (i) the permissioned personal genetic profile of the user and (ii) the permissioned personal genetic profile of the member; and determining the member to be a potential match based at least in part on the computed compatibility index for the member and the user. In certain embodiments, step (c) comprises determining the member to be a potential match based at least in part on at least a portion of a biographical profile of the member.

In certain embodiments, method comprises: presenting, by the processor, a profile builder graphical user interface (GUI) for user selection and/or updating of permissioned personal genetic profile content; receiving, by the processor, via the profile builder GUI, a user selection of one or more accessible genetic profile objects (e.g., genetic interest objects; e.g., variant objects; e.g., secondary profile tags) to include; and storing, by the processor, within the permissioned personal genetic profile of the user, the user selection of the one or more accessible genetic profile objects to include.

In certain embodiments, the method comprises: accessing, by the processor, the permissioned personal genetic profile of the user; presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receiving, by the processor, via the profile builder GUI, a user selection of one or more accessible genetic profile objects (e.g., genetic interest objects; e.g., variant objects; e.g., secondary profile tags) to exclude from their personal genetic profile; and removing, by the processor, from the permissioned personal genetic profile of the user, the one or more accessible genetic profile objects selected by the user for exclusion from their permissioned personal genetic profile.

In another aspect, the invention is directed to a method of automatically identifying and presenting to a user, one or more nearby genetic profile matches based on shared genetic interests and/or similar (and/or compatible) genotypes via a graphical user interface (GUI), the method comprising: (a) identifying, by the processor, one or more nearby members of a genetic profile based matching service in geographic proximity to the user based on location data (e.g., GPS data) for the user and location data (e.g., GPS data) for each of the one or more members; (b) accessing, by a processor of a computing device, a permissioned personal genetic profile of the user that identifies genetic interests of the user (e.g., preferred genetic traits that the user would like to find in a nearby match, and/or preferred categories of traits with which to compare the user with other members of genetic profile based matching service for identification of a nearby genetic profile match for the user) and/or genetic information to which the user has permitted access by the processor; (c) accessing, by the processor, permissioned personal genetic profiles of each of one or more of the nearby members; (d) for each of a subset of the one or more nearby members, determining, by the processor, the member to be a genetic profile match based on the permissioned personal genetic profile of the user and the permissioned personal genetic profile of the member, thereby automatically identifying one or more nearby genetic profile match(es) for the user; and (e) causing, by the processor, graphical rendering of, for presentation to the user within the GUI (e.g., for presentation on a user's mobile computing device) graphics and/or text corresponding to at least a portion of a biographical profile of each of the one or more nearby genetic profile match(es).

In certain embodiments, step (e) comprises causing graphical rendering of the graphics and/or text corresponding to the at least a portion of the biographical profile of each of the one or more nearby genetic profile match(es) in a location dependent fashion within the GUI (e.g., so as to indicate a location of each nearby match with respect to the user (e.g., on a map)) based on the location data (e.g., GPS coordinates) for each of the one or more nearby match(es) and the location data for the user.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in a potential match with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, for each of the one or more nearby members (e.g., up to all) of the genetic profile based matching service, the permissioned personal genetic profile of the nearby member comprises one or more genetic interest identifiers (e.g., textual labels) for the nearby member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of the one or more nearby members (e.g., up to all) of the genetic profile based matching service, the permissioned personal genetic profile of the nearby member comprises one or more accessible variant objects for the nearby member, each representing a particular variant of a particular SNP that the nearby member has (e.g., as determined via genotyping measurements) (e.g., each accessible variant object comprising a measurement outcome that identifies the particular variant that the nearby member has and/or a qualifier that classifies the particular variant that the nearby member has), and to which the nearby member has granted access.

In certain embodiments, for each of the one or more nearby members (e.g., up to all) of the genetic profile based matching service, the permissioned personal genetic profile of the nearby member comprises one or more secondary profile tags (e.g., dietary profile tags; e.g., physical fitness tags) for the nearby member, the secondary profile tags having been determined based on genotyping data for the nearby member.

In certain embodiments, for each of the one or more nearby members (e.g., up to all) of the genetic profile based matching service, the permissioned personal genetic profile of the nearby member comprises one or more member-specified weighting values, each associated with a specific accessible genetic profile object (e.g., a genetic interest indicator, accessible variant object, or secondary profile tag) of the permissioned personal genetic profile of the nearby member and indicating a level of desired similarity and/or dissimilarity and/or compatibility in a genetic profile match with respect to the specific accessible genetic profile object of the permissioned personal genetic profile of the nearby member (e.g., the genetic interest indicator, accessible variant object, or secondary profile tag).

In certain embodiments, step (d) comprises, for each of one or more nearby members of the genetic profile based matching service: computing a compatibility index for the nearby member and the user based at least in part on (i) the permissioned personal genetic profile of the user and (ii) the permissioned personal genetic profile of the nearby member; and determining the nearby member to be a genetic profile match based at least in part on the computed compatibility index for the nearby member and the user.

In certain embodiments, the method comprises: presenting, by the processor, a profile builder graphical user interface (GUI) for user selection and/or updating of permissioned personal genetic profile content; receiving, by the processor, via the profile builder GUI, a user selection of one or more accessible genetic profile objects (e.g., genetic interest objects; e.g., variant objects; e.g., secondary profile tags) to include; and storing, by the processor, within the permissioned personal genetic profile of the user, the user selection of the one or more accessible genetic profile objects to include.

In certain embodiments, the method comprises: accessing, by the processor, the permissioned personal genetic profile of the user; presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receiving, by the processor, via the profile builder GUI, a user selection of one or more accessible genetic profile objects (e.g., genetic interest objects; e.g., variant objects; e.g., secondary profile tags) to exclude from their personal genetic profile; and removing, by the processor, from the permissioned personal genetic profile of the user, the one or more accessible genetic profile objects selected by the user for exclusion from their permissioned personal genetic profile.

In another aspect, the invention is directed to a method of automatically identifying and presenting to a user, genetically tailored online content via a graphical user interface (GUI), the method comprising: (a) accessing, by a processor of a computing device, a permissioned personal genetic profile of the user that identifies genetic interests of the user (e.g., genetic traits and/or categories of traits for which the user would like to see online content of relevance) and/or genetic information to which the user has permitted access by the processor; (b) accessing, by the processor, genetic profiles of each of one or more content objects; (c) for each of a subset of the one or more content objects, determining, by the processor, the content object to be genetically relevant to the user based on the permissioned personal genetic profile of the user and the genetic profile of the content object, thereby automatically identifying one or more genetically relevant content objects for the user; and (d) causing, by the processor, graphical rendering of, for presentation to the user within the GUI (e.g., for presentation on a user's mobile computing device) graphics and/or text corresponding to renderable data of the one or more genetically relevant content objects.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in genetic profiles associated with content objects with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, each of at least a portion (e.g., up to all) of the one or more the content objects is a member post to an online forum (e.g., a social media platform; e.g., an online message board), associated with a particular member (e.g., the member who posted the post) and, for each member post, the genetic profile of the member post is a permissioned personal genetic profile of the particular member with which the post is associated (e.g., the member that posted the post).

In certain embodiments, for each member post, the permissioned personal genetic profile of the particular member with which the member post is associated comprises one or more genetic interest identifiers (e.g., textual labels) for the particular member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each member post, the permissioned personal genetic profile of the particular member with which the member post is associated comprises one or more accessible variant objects for the particular member, each representing a particular variant of a particular SNP that the particular member has (e.g., as determined via genotyping measurements) (e.g., each accessible variant object comprising a measurement outcome that identifies the particular variant that the particular member has and/or a qualifier that classifies the particular variant that the particular member has), and to which the particular member has granted access.

In certain embodiments, for each member post, the permissioned personal genetic profile of the particular member with which the member post is associated comprises one or more secondary profile tags (e.g., dietary profile tags; e.g., physical fitness tags) for the particular member, the secondary profile tags having been determined based on genotyping data for the particular member.

In certain embodiments, for each member post, the permissioned personal genetic profile of the particular member with which the member post is associated comprises one or more member-specified weighting values, each associated with a specific accessible genetic profile object (e.g., a genetic interest indicator, accessible variant object, or secondary profile tag) of the permissioned personal genetic profile of the particular member and indicating a level of similarity and/or dissimilarity and/or compatibility desired by the particular member in genetically relevant content that they view with respect to the specific accessible genetic profile object of the permissioned personal genetic profile of the particular member (e.g., the genetic interest indicator, accessible variant object, or secondary profile tag).

In certain embodiments, step (c) comprises, for each member post: computing a compatibility index for the particular member with which the member post is associated and the user based at least in part on (i) the permissioned personal genetic profile of the user and (ii) the permissioned personal genetic profile of the particular member; and determining the member post to be genetically relevant based at least in part on the computed compatibility index for the particular member and the user.

In certain embodiments, at least a portion of the one or more content objects are commercial posts (e.g., sponsored posts created as advertisement; e.g., posts from a news media outlet), each having a content-specific genetic profile comprising one or more content-specific genetic profile objects (e.g., assigned by a developer; e.g., generated and assigned automatically, by the processor, based text that the commercial post comprises) that identify genetic interests for which the commercial post is relevant and/or genetic information (e.g., identifications of particular variants of particular SNPs) that identifies a subset of genotypes for which the commercial post is relevant.

In certain embodiments, for each commercial post, the content-specific genetic profile associated with the commercial post comprises one or more genetic interest identifiers (e.g., textual labels) (e.g., assigned by a developer; e.g., automatically generated based on content of the commercial post), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each commercial post, the content-specific genetic profile associated with the commercial post comprises one or more content-specific variant objects (e.g., assigned by a developer; e.g., automatically generated based on content of the commercial post), each representing a particular variant of a particular SNP that identifies a subset of genotypes for which the commercial post is relevant.

In certain embodiments, for each commercial post, the content-specific genetic profile of the commercial post comprises one or more content-specific secondary profile tags (e.g., dietary profile tags; e.g., physical fitness tags) (e.g., assigned by a developer; e.g., automatically generated based on content of the commercial post) that are matchable to a viable secondary profile tags of a permissioned personal genetic profile (e.g., of the user and/or one or more members of an online forum).

In certain embodiments, for each commercial post, the content specific genetic profile of the commercial post comprises one or more content-specific weighting values, each associated with a particular content-specific genetic profile object (e.g., a genetic interest indicator, content-specific variant object, or secondary profile tag) of the content-specific genetic profile and indicating a level similarity and/or dissimilarity and/or compatibility of the commercial post with the user (e.g., and/or other members of an online forum) with respect to the particular content-specific genetic profile object of the content-specific genetic profile of the commercial post (e.g., the genetic interest identifier, content-specific variant object, or content-specific secondary profile tag).

In certain embodiments, step (c) comprises, for each commercial post: computing a compatibility index for the commercial post and the user based at least in part on (i) the permissioned personal genetic profile of the user and (ii) the content-specific genetic profile associated with the commercial post; and determining the commercial post to be genetically relevant based at least in part on the computed compatibility index for the commercial post and the user.

In certain embodiments, the method comprises: presenting, by the processor, a profile builder graphical user interface (GUI) for user selection and/or updating of permissioned personal genetic profile content; receiving, by the processor, via the profile builder GUI, a user selection of one or more accessible genetic profile objects (e.g., genetic interest objects; e.g., variant objects; e.g., secondary profile tags) to include; and storing, by the processor, within the permissioned personal genetic profile of the user, the user selection of the one or more accessible genetic profile objects to include.

In certain embodiments, the method comprises: accessing, by the processor, the permissioned personal genetic profile of the user; presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receiving, by the processor, via the profile builder GUI, a user selection of one or more accessible genetic profile objects (e.g., genetic interest objects; e.g., variant objects; e.g., secondary profile tags) to exclude from their personal genetic profile; and removing, by the processor, from the permissioned personal genetic profile of the user, the one or more accessible genetic profile objects selected by the user for exclusion from their permissioned personal genetic profile.

In another aspect, the invention is directed to a method of automatically managing and filtering social media interactions of a user with other social media platform members based on shared genetic interests and/or personal genetic data, the method comprising: (a) accessing, by a processor of a computing device, a permissioned personal genetic profile of the user that identifies genetic interests of the user and/or genetic information that they have shared; (b) accessing, by the processor, permissioned personal genetic profiles of each of one or more social media platform members; (c) for each of a subset of the one or more social media members, determining, by the processor, the member to be a genetic profile match to the user based on the permissioned personal genetic profile of the user and the permissioned personal genetic profile of the social media platform member, thereby automatically identifying one or more genetic profile matches for the user; and (d) causing, by the processor, graphical rendering of, for presentation to the user, within a graphical user interface (GUI), graphics and/or text corresponding to (i) at least a portion of a biographical profile of each of the one or more genetic profile matches for the user and/or (ii) set of renderable data associated with a content object associated (e.g., posted by) each of at least a portion of the one or more genetic profile matches.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in member of the social media platform determined to be a genetic profile match with respect to the accessible genetic profile object (e.g., the genetic interest indicator, accessible variant object, or secondary profile tag) of the user permissioned personal genetic profile (e.g., select forced match categories, weighted categories, etc.).

In certain embodiments, for each of the one or more social media platform members (e.g., up to all), the permissioned personal genetic profile of the social media platform member comprises one or more genetic interest identifiers (e.g., textual labels) for the social media platform member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of the one or more social media platform members (e.g., up to all), the permissioned personal genetic profile of the social media platform member comprises one or more accessible variant objects for the social media platform member, each representing a particular variant of a particular SNP that the social media platform member has (e.g., as determined via genotyping measurements) (e.g., each accessible variant object comprising a measurement outcome that identifies the particular variant that the social media platform member has and/or a qualifier that classifies the particular variant that the social media platform member has), and to which the social media platform member has granted access.

In certain embodiments, for each of the one or more social media platform members (e.g., up to all), the permissioned personal genetic profile of the social media platform member comprises one or more secondary profile tags (e.g., dietary profile tags; e.g., physical fitness tags) for the social media platform member, the secondary profile tags having been determined based on genotyping data for the social media platform member.

In certain embodiments, for each of the one or more social media platform members (e.g., up to all), the permissioned personal genetic profile of the social media platform member comprises one or more member-specified weighting values, each associated with a specific accessible genetic profile object (e.g., a genetic interest indicator, accessible variant object, or secondary profile tag) of the permissioned personal genetic profile of the social media platform member and indicating a level of desired similarity and/or dissimilarity and/or compatibility in a genetic profile match with respect to the specific accessible genetic profile object of the permissioned personal genetic profile of the social media platform member (e.g., the genetic interest indicator, accessible variant object, or secondary profile tag).

In certain embodiments, step (c) comprises, for each of one or more social media platform members: computing a compatibility index for the social media platform member and the user based at least in part on (i) the permissioned personal genetic profile of the user and (ii) the permissioned personal genetic profile of the social media platform member; and determining the social media platform member to be a genetic profile match based at least in part on the computed compatibility index for the social media platform member and the user.

In certain embodiments, the method comprises: presenting, by the processor, a profile builder graphical user interface (GUI) for user selection and/or updating of permissioned personal genetic profile content; receiving, by the processor, via the profile builder GUI, a user selection of one or more accessible genetic profile objects (e.g., genetic interest objects; e.g., variant objects; e.g., secondary profile tags) to include; and storing, by the processor, within the permissioned personal genetic profile of the user, the user selection of the one or more accessible genetic profile objects to include.

In certain embodiments, the method comprises: accessing, by the processor, the permissioned personal genetic profile of the user; presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receiving, by the processor, via the profile builder GUI, a user selection of one or more accessible genetic profile objects (e.g., genetic interest objects; e.g., variant objects; e.g., secondary profile tags) to exclude from their personal genetic profile; and removing, by the processor, from the permissioned personal genetic profile of the user, the one or more accessible genetic profile objects selected by the user for exclusion from their permissioned personal genetic profile.

In certain embodiments, step (d) comprises, for each set of renderable data associated with a content object, causing rendering of a graphics identifying one or more common genetic interests and/or traits between the user and the genetic profile match associated with the content object, each common genetic interest and/or trait corresponding to an accessible genetic profile object of the permissioned personal genetic profile of the genetic profile match that matches (e.g., represents a same gene, SNP, variant, and/or qualifier) an accessible genetic profile object of the permissioned personal genetic profile of the user.

In certain embodiments, the graphics identifying one or more common genetic interest and/or traits comprise, for each matching accessible genetic profile object, a graphical representation of a gene associated with both (i) the accessible genetic profile object of the permissioned personal genetic profile of the genetic profile match and (ii) the genetic profile object of the permissioned personal genetic profile of the user.

In certain embodiments, the method comprises: receiving (e.g., via a member lookup view of the GUI), by the processor, a user input to identify genetic profile matches (e.g., a user click on a graphical control element (e.g., a selectable button) of the member lookup view and/or entry of text into a lookup query dialog); and performing steps (a)-(d) responsive to receipt of the user input to identify genetic profile matches.

In certain embodiments, the user input to identify genetic profile matches comprises a lookup query (e.g., entered into a lookup query dialog), and wherein step (c) further comprises matching at least a portion of the lookup query to data (e.g., a member identifier; e.g., a member name; e.g., a brief biography of the member) stored in the biographical profile of each of the one or more genetic profile matches, such that the social media platform members are determined to be genetic profile matches of the user based further on the matching between the portion of the lookup query and the data stored in their biographical profiles. In certain embodiments, the lookup query comprises one or more of: (i) a member identifier [e.g., text that identifies a member (e.g., a user-id or nickname included in a member biographical profile)], (ii) a member name, and (iii) searchable text (e.g., one or more keywords/keyphrases).

In certain embodiments, step (d) comprises: receiving, by the processor, a user input corresponding to a selection of at least a portion of the one or more genetic profile matches for the user for addition to a list of followed members [e.g., via a user selection of one or more selectable buttons (e.g., “follow” buttons), each associated with a genetic profile match and displayed in the GUI]; adding, by the processor, the selected genetic profile matches to the list of followed members, thereby identifying the selected genetic profile matches as followed members; and causing, by the processor, graphical rendering of, for presentation to the user, (e.g., within a user feed window of the GUI), graphics and/or text corresponding to a set of renderable data associated with a content object associated with (e.g., posted by) each of at least a portion of the followed members.

In another aspect, the invention is directed to a system for automatically identifying, presenting, and matching a user to members of a dating pool based on genetic interests and/or shared genetic information via a graphical user interface (GUI), the system comprising: a processor of a computing device; and a memory having instructions stored thereon, wherein the instructions, when executed by the processor, cause the processor to: (a) access a permissioned personal genetic profile of the user that identifies genetic interests of the user (e.g., preferred genetic traits of a potential match for the user, and/or preferred categories of traits with which to compare the user with other members of the data pool for identification of a potential match for the user) and/or genetic information to which the user has permitted access by the processor; (b) access permissioned personal genetic profiles of each of one or more of the members of the dating pool; (c) for each of a subset of the one or more members of the dating pool, determine the member to be a potential match based at least in part on the permissioned personal genetic profile of the user and the permissioned personal genetic profile of the member, thereby automatically identifying one or more potential matches for the user; and (d) cause graphical rendering of, for presentation to the user within the GUI (e.g., for presentation on a user's mobile computing device), graphics and/or text corresponding to at least a portion of a biographical profile of each of the one or more potential matches.

In certain embodiments, at least one of the potential matches is linked to a first interest indicator corresponding to the user and having an affirmative value, and the instructions cause the processor to: (e) receive, via the GUI, a user input corresponding to second interest indicator corresponding to the at least one potential match and having an affirmative value; (f) determine, the at least one potential match to be a positive match for the user based on both the first and second interest indicators having affirmative values; and (g) responsive to the determining the at least one potential match to be a positive match for the user, issue a notification to the user of the positive match.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in a potential match with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, for each of one or more members (e.g., up to all) of the dating pool, the permissioned personal genetic profile of the member comprises one or more genetic interest identifiers (e.g., textual labels) for the member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of one or more members (e.g., up to all) of the dating pool, the permissioned personal genetic profile of the member comprises one or more accessible variant objects for the member, each representing a particular variant of a particular SNP that the member has (e.g., as determined via genotyping measurements) (e.g., each accessible variant object comprising a measurement outcome that identifies the particular variant that the member has and/or a qualifier that classifies the particular variant that the member has), and to which the member has granted access.

In certain embodiments, for each of one or more members (e.g., up to all) of the dating pool, the permissioned personal genetic profile of the member comprises one or more secondary profile tags (e.g., dietary profile tags; e.g., physical fitness tags) for the member, the secondary profile tags having been determined based on genotyping data for the member.

In certain embodiments, for each of one or more members (e.g., up to all) of the dating pool, the permissioned personal genetic profile of the member comprises one or more member-specified weighting values, each associated with a specific accessible genetic profile object (e.g., a genetic interest indicator, accessible variant object, or secondary profile tag) of the permissioned personal genetic profile of the member and indicating a level of desired similarity and/or dissimilarity and/or compatibility in a potential match with respect to the specific accessible genetic profile object of the permissioned personal genetic profile of the member (e.g., the genetic interest indicator, accessible variant object, or secondary profile tag).

In certain embodiments, at step (c), the instructions cause the processor to, for each of one or more members of the dating pool: compute a compatibility index for the member and the user based at least in part on (i) the permissioned personal genetic profile of the user and (ii) the permissioned personal genetic profile of the member; and determine the member to be a potential match based at least in part on the computed compatibility index for the member and the user.

In certain embodiments, at step (c), the instructions cause the processor to determine the member to be a potential match based at least in part on at least a portion of a biographical profile of the member.

In certain embodiments, the instructions cause the processor to: present a profile builder graphical user interface (GUI) for user selection and/or updating of permissioned personal genetic profile content; receive, via the profile builder GUI, a user selection of one or more accessible genetic profile objects (e.g., genetic interest objects; e.g., variant objects; e.g., secondary profile tags) to include; and store within the permissioned personal genetic profile of the user, the user selection of the one or more accessible genetic profile objects to include.

In certain embodiments, the instructions cause the processor to: access the permissioned personal genetic profile of the user; present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receive, via the profile builder GUI, a user selection of one or more accessible genetic profile objects (e.g., genetic interest objects; e.g., variant objects; e.g., secondary profile tags) to exclude from their personal genetic profile; and remove, from the permissioned personal genetic profile of the user, the one or more accessible genetic profile objects selected by the user for exclusion from their permissioned personal genetic profile.

In another aspect, the invention is directed to a system for automatically identifying and presenting to a user, one or more nearby genetic profile matches based on shared genetic interests and/or similar (and/or compatible) genotypes via a graphical user interface (GUI), the system comprising: a processor of a computing device; and a memory having instructions stored thereon, wherein the instructions, when executed by the processor, cause the processor to: (a) identify one or more nearby members of a genetic profile based matching service in geographic proximity to the user based on location data (e.g., GPS data) for the user and location data (e.g., GPS data) for each of the one or more members; (b) access a permissioned personal genetic profile of the user that identifies genetic interests of the user (e.g., preferred genetic traits that the user would like to find in a nearby match, and/or preferred categories of traits with which to compare the user with other members of genetic profile based matching service for identification of a nearby genetic profile match for the user) and/or genetic information to which the user has permitted access by the processor; (c) access permissioned personal genetic profiles of each of one or more of the nearby members; (d) for each of a subset of the one or more nearby members, determine the member to be a genetic profile match based on the permissioned personal genetic profile of the user and the permissioned personal genetic profile of the member, thereby automatically identifying one or more nearby genetic profile match(es) for the user; and (e) cause graphical rendering of, for presentation to the user within the GUI (e.g., for presentation on a user's mobile computing device) graphics and/or text corresponding to at least a portion of a biographical profile of each of the one or more nearby genetic profile match(es).

In certain embodiments, at step (e), the instructions cause the processor to cause graphical rendering of the graphics and/or text corresponding to the at least a portion of the biographical profile of each of the one or more nearby genetic profile match(es) in a location dependent fashion within the GUI (e.g., so as to indicate a location of each nearby match with respect to the user (e.g., on a map)) based on the location data (e.g., GPS coordinates) for each of the one or more nearby match(es) and the location data for the user.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in a potential match with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, for each of the one or more nearby members (e.g., up to all) of the genetic profile based matching service, the permissioned personal genetic profile of the nearby member comprises one or more genetic interest identifiers (e.g., textual labels) for the nearby member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of the one or more nearby members (e.g., up to all) of the genetic profile based matching service, the permissioned personal genetic profile of the nearby member comprises one or more accessible variant objects for the nearby member, each representing a particular variant of a particular SNP that the nearby member has (e.g., as determined via genotyping measurements) (e.g., each accessible variant object comprising a measurement outcome that identifies the particular variant that the nearby member has and/or a qualifier that classifies the particular variant that the nearby member has), and to which the nearby member has granted access.

In certain embodiments, for each of the one or more nearby members (e.g., up to all) of the genetic profile based matching service, the permissioned personal genetic profile of the nearby member comprises one or more secondary profile tags (e.g., dietary profile tags; e.g., physical fitness tags) for the nearby member, the secondary profile tags having been determined based on genotyping data for the nearby member.

In certain embodiments, for each of the one or more nearby members (e.g., up to all) of the genetic profile based matching service, the permissioned personal genetic profile of the nearby member comprises one or more member-specified weighting values, each associated with a specific accessible genetic profile object (e.g., a genetic interest indicator, accessible variant object, or secondary profile tag) of the permissioned personal genetic profile of the nearby member and indicating a level of desired similarity and/or dissimilarity and/or compatibility in a genetic profile match with respect to the specific accessible genetic profile object of the permissioned personal genetic profile of the nearby member (e.g., the genetic interest indicator, accessible variant object, or secondary profile tag).

In certain embodiments, at step (d), the instructions cause the processor to, for each of one or more nearby members of the genetic profile based matching service: compute a compatibility index for the nearby member and the user based at least in part on (i) the permissioned personal genetic profile of the user and (ii) the permissioned personal genetic profile of the nearby member; and determine the nearby member to be a genetic profile match based at least in part on the computed compatibility index for the nearby member and the user.

In certain embodiments, the instructions cause the processor to: present a profile builder graphical user interface (GUI) for user selection and/or updating of permissioned personal genetic profile content; receive, via the profile builder GUI, a user selection of one or more accessible genetic profile objects (e.g., genetic interest objects; e.g., variant objects; e.g., secondary profile tags) to include; and storing, by the processor, within the permissioned personal genetic profile of the user, the user selection of the one or more accessible genetic profile objects to include.

In certain embodiments, the instructions cause the processor to: access the permissioned personal genetic profile of the user; present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receive, via the profile builder GUI, a user selection of one or more accessible genetic profile objects (e.g., genetic interest objects; e.g., variant objects; e.g., secondary profile tags) to exclude from their personal genetic profile; and remove, from the permissioned personal genetic profile of the user, the one or more accessible genetic profile objects selected by the user for exclusion from their permissioned personal genetic profile.

In another aspect, the invention is directed to a system for automatically identifying and presenting to a user, genetically tailored online content via a graphical user interface (GUI), the system comprising: a processor of a computing device; and a memory having instructions stored thereon, wherein the instructions, when executed by the processor, cause the processor to: (a) access a permissioned personal genetic profile of the user that identifies genetic interests of the user (e.g., genetic traits and/or categories of traits for which the user would like to see online content of relevance) and/or genetic information to which the user has permitted access by the processor; (b) access genetic profiles of each of one or more content objects; (c) for each of a subset of the one or more content objects, determine the content object to be genetically relevant to the user based on the permissioned personal genetic profile of the user and the genetic profile of the content object, thereby automatically identifying one or more genetically relevant content objects for the user; and (d) cause graphical rendering of, for presentation to the user within the GUI (e.g., for presentation on a user's mobile computing device) graphics and/or text corresponding to renderable data of the one or more genetically relevant content objects.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility in genetic profiles associated with content objects with respect to the specific accessible genetic profile object (e.g., the genetic interest indicator, accessible variant object, or secondary profile tag) of the user permissioned personal genetic profile (e.g., select forced match categories, weighted categories, etc.).

In certain embodiments, each of at least a portion (e.g., up to all) of the one or more the content objects is a member post to an online forum (e.g., a social media platform; e.g., an online message board), associated with a particular member (e.g., the member who posted the post) and, for each member post, the genetic profile of the member post is a permissioned personal genetic profile of the particular member with which the post is associated (e.g., the member that posted the post).

In certain embodiments, for each member post, the permissioned personal genetic profile of the particular member with which the member post is associated comprises one or more genetic interest identifiers (e.g., textual labels) for the particular member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each member post, the permissioned personal genetic profile of the particular member with which the member post is associated comprises one or more accessible variant objects for the particular member, each representing a particular variant of a particular SNP that the particular member has (e.g., as determined via genotyping measurements) (e.g., each accessible variant object comprising a measurement outcome that identifies the particular variant that the particular member has and/or a qualifier that classifies the particular variant that the particular member has), and to which the particular member has granted access.

In certain embodiments, for each member post, the permissioned personal genetic profile of the particular member with which the member post is associated comprises one or more secondary profile tags (e.g., dietary profile tags; e.g., physical fitness tags) for the particular member, the secondary profile tags having been determined based on genotyping data for the particular member.

In certain embodiments, for each member post, the permissioned personal genetic profile of the particular member with which the member post is associated comprises one or more member-specified weighting values, each associated with a specific accessible genetic profile object (e.g., a genetic interest indicator, accessible variant object, or secondary profile tag) of the permissioned personal genetic profile of the particular member and indicating a level of similarity and/or dissimilarity and/or compatibility desired by the particular member in genetically relevant content that they view with respect to the specific accessible genetic profile object of the permissioned personal genetic profile of the particular member (e.g., the genetic interest indicator, accessible variant object, or secondary profile tag).

In certain embodiments, at step (c) the instructions cause the processor to, for each member post: compute a compatibility index for the particular member with which the member post is associated and the user based at least in part on (i) the permissioned personal genetic profile of the user and (ii) the permissioned personal genetic profile of the particular member; and determine the member post to be genetically relevant based at least in part on the computed compatibility index for the particular member and the user.

In certain embodiments, at least a portion of the one or more content objects are commercial posts (e.g., sponsored posts created as advertisement; e.g., posts from a news media outlet), each having a content-specific genetic profile comprising one or more content-specific genetic profile objects (e.g., assigned by a developer; e.g., generated and assigned automatically, by the processor, based text that the commercial post comprises) that identify genetic interests for which the commercial post is relevant and/or genetic information (e.g., identifications of particular variants of particular SNPs) that identifies a subset of genotypes for which the commercial post is relevant.

In certain embodiments, for each commercial post, the content-specific genetic profile associated with the commercial post comprises one or more genetic interest identifiers (e.g., textual labels) (e.g., assigned by a developer; e.g., automatically generated based on content of the commercial post), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each commercial post, the content-specific genetic profile associated with the commercial post comprises one or more content-specific variant objects (e.g., assigned by a developer; e.g., automatically generated based on content of the commercial post), each representing a particular variant of a particular SNP that identifies a subset of genotypes for which the commercial post is relevant.

In certain embodiments, for each commercial post, the content-specific genetic profile of the commercial post comprises one or more content-specific secondary profile tags (e.g., dietary profile tags; e.g., physical fitness tags) (e.g., assigned by a developer; e.g., automatically generated based on content of the commercial post) that are matchable to viable secondary profile tags of a permissioned personal genetic profile (e.g., of the user and/or one or more members of an online forum).

In certain embodiments, for each commercial post, the content specific genetic profile of the commercial post comprises one or more content-specific weighting values, each associated with a particular content-specific genetic profile object (e.g., a genetic interest indicator, content-specific variant object, or secondary profile tag) of the content-specific genetic profile and indicating a level similarity and/or dissimilarity and/or compatibility of the commercial post with the user (e.g., and/or other members of an online forum) with respect to the particular content-specific genetic profile object of the content-specific genetic profile of the commercial post (e.g., the genetic interest identifier, content-specific variant object, or content-specific secondary profile tag).

In certain embodiments, at step (c), the instructions cause the processor to, for each commercial post: compute a compatibility index for the commercial post and the user based at least in part on (i) the permissioned personal genetic profile of the user and (ii) the content-specific genetic profile associated with the commercial post; and determine the commercial post to be genetically relevant based at least in part on the computed compatibility index for the commercial post and the user.

In certain embodiments, the instructions cause the processor to: present a profile builder graphical user interface (GUI) for user selection and/or updating of permissioned personal genetic profile content; receive via the profile builder GUI, a user selection of one or more accessible genetic profile objects (e.g., genetic interest objects; e.g., variant objects; e.g., secondary profile tags) to include; and store, within the permissioned personal genetic profile of the user, the user selection of the one or more accessible genetic profile objects to include.

In certain embodiments, the instructions cause the processor to: access the permissioned personal genetic profile of the user; present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receive, via the profile builder GUI, a user selection of one or more accessible genetic profile objects (e.g., genetic interest objects; e.g., variant objects; e.g., secondary profile tags) to exclude from their personal genetic profile; and remove, from the permissioned personal genetic profile of the user, the one or more accessible genetic profile objects selected by the user for exclusion from their permissioned personal genetic profile.

In another aspect, the invention is directed to a system for automatically managing and filtering social media interactions of a user with other social media platform members based on shared genetic interests and/or personal genetic data, the system comprising: a processor of a computing device; and a memory having instructions stored thereon, wherein the instructions, when executed by the processor, cause the processor to: (a) access a permissioned personal genetic profile of the user that identifies genetic interests of the user and/or genetic information that they have shared; (b) access permissioned personal genetic profiles of each of one or more social media platform members; (c) for each of a subset of the one or more social media platform members, determine the member to be a genetic profile match to the user based on the permissioned personal genetic profile of the user and the permissioned personal genetic profile of the social media platform member, thereby automatically identifying one or more genetic profile matches for the user; and (d) cause graphical rendering of, for presentation to the user, within a graphical user interface (GUI), graphics and/or text corresponding to (i) at least a portion of a biographical profile of each of the one or more genetic profile matches for the user and/or (ii) set of renderable data associated with a content object associated (e.g., posted by) each of at least a portion of the one or more genetic profile matches.

In certain embodiments, the permissioned personal genetic profile of the user comprises one or more genetic interest identifiers (e.g., textual labels), each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, the permissioned personal genetic profile of the user comprises one or more accessible variant objects, each representing a particular variant of a particular SNP that the user has (e.g., as determined via genotyping measurements) (e.g., each accessible variant object comprising a measurement outcome that identifies the particular variant that the user has and/or a qualifier that classifies the particular variant that the user has), and to which the user has granted access.

In certain embodiments, the permissioned personal genetic profile of the user comprises one or more secondary profile tags (e.g., dietary profile tags; e.g., physical fitness tags) having been determined based on genotyping data for the user.

In certain embodiments, the permissioned personal genetic profile of the user comprises one or more user-specified weighting values, each associated with a specific accessible genetic profile object (e.g., a genetic interest indicator, accessible variant object, or secondary profile tag) of the user permissioned personal genetic profile and indicating a level of desired similarity and/or dissimilarity and/or compatibility with members of the social media platform determined to be a genetic profile match with respect to the accessible genetic profile object (e.g., the genetic interest indicator, accessible variant object, or secondary profile tag) of the user permissioned personal genetic profile (e.g., select forced match categories, weighted categories, etc.).

In certain embodiments, for each of the one or more social media platform members (e.g., up to all), the permissioned personal genetic profile of the social media platform member comprises one or more genetic interest identifiers (e.g., textual labels) for the social media platform member, each corresponding to (e.g., identifying) a stored genomic object (e.g., a product; e.g., a category; e.g., a gene object; e.g., a SNP object; e.g., a variant object) (e.g., such that each genetic interest identifier represents either (i) a single particular SNP or variant thereof or (ii) a set of one or more SNPs).

In certain embodiments, for each of the one or more social media platform members (e.g., up to all), the permissioned personal genetic profile of the social media platform member comprises one or more accessible variant objects for the social media platform member, each representing a particular variant of a particular SNP that the social media platform member has (e.g., as determined via genotyping measurements) (e.g., each accessible variant object comprising a measurement outcome that identifies the particular variant that the social media platform member has and/or a qualifier that classifies the particular variant that the social media platform member has), and to which the social media platform member has granted access.

In certain embodiments, for each of the one or more social media platform members (e.g., up to all), the permissioned personal genetic profile of the social media platform member comprises one or more secondary profile tags (e.g., dietary profile tags; e.g., physical fitness tags) for the social media platform member, the secondary profile tags having been determined based on genotyping data for the social media platform member.

In certain embodiments, for each of the one or more social media platform members (e.g., up to all), the permissioned personal genetic profile of the social media platform member comprises one or more member-specified weighting values, each associated with a specific accessible genetic profile object (e.g., a genetic interest indicator, accessible variant object, or secondary profile tag) of the permissioned personal genetic profile of the social media platform member and indicating a level of desired similarity and/or dissimilarity and/or compatibility in a genetic profile match with respect to the specific accessible genetic profile object of the permissioned personal genetic profile of the social media platform member (e.g., the genetic interest indicator, accessible variant object, or secondary profile tag).

In certain embodiments, at step (c), the instructions cause the processor to, for each of one or more social media platform members: compute a compatibility index for the social media platform member and the user based at least in part on (i) the permissioned personal genetic profile of the user and (ii) the permissioned personal genetic profile of the social media platform member; and determine the social media platform member to be a genetic profile match based at least in part on the computed compatibility index for the social media platform member and the user.

In certain embodiments, the instructions cause the processor to: present a profile builder graphical user interface (GUI) for user selection and/or updating of permissioned personal genetic profile content; receive, via the profile builder GUI, a user selection of one or more accessible genetic profile objects (e.g., genetic interest objects; e.g., variant objects; e.g., secondary profile tags) to include; and store, within the permissioned personal genetic profile of the user, the user selection of the one or more accessible genetic profile objects to include.

In certain embodiments, the instructions cause the processor to: access the permissioned personal genetic profile of the user; present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object; receive, via the profile builder GUI, a user selection of one or more accessible genetic profile objects (e.g., genetic interest objects; e.g., variant objects; e.g., secondary profile tags) to exclude from their personal genetic profile; and remove, from the permissioned personal genetic profile of the user, the one or more accessible genetic profile objects selected by the user for exclusion from their permissioned personal genetic profile.

In certain embodiments, at step (d), the instructions cause the processor to, for each set of renderable data associated with a content object, causing rendering of a graphics identifying one or more common genetic interests and/or traits between the user and the genetic profile match associated with the content object, each common genetic interest and/or trait corresponding to an accessible genetic profile object of the permissioned personal genetic profile of the genetic profile match that matches (e.g., represents a same gene, SNP, variant, and/or qualifier) an accessible genetic profile object of the permissioned personal genetic profile of the user.

In certain embodiments, the graphics identifying one or more common genetic interest and/or traits comprise, for each matching accessible genetic profile object, a graphical representation of a gene associated with both (i) the accessible genetic profile object of the permissioned personal genetic profile of the genetic profile match and (ii) the genetic profile object of the permissioned personal genetic profile of the user.

In certain embodiments, the instructions cause the processor to: receive (e.g., via a member lookup view of the GUI) a user input to identify genetic profile matches (e.g., a user click on a graphical control element (e.g., a selectable button) of the member lookup view and/or entry of text into a lookup query dialog); and responsive to receipt of the user input to identify genetic profile matches, perform steps (a)-(d).

In certain embodiments, the user input to identify genetic profile matches comprises a lookup query (e.g., entered into a lookup query dialog), and wherein step (c) further comprises matching at least a portion of the lookup query to data (e.g., a member identifier; e.g., a member name; e.g., a brief biography of the member) stored in the biographical profile of each of the one or more genetic profile matches, such that the social media platform members are determined to be genetic profile matches of the user based further on the matching between the portion of the lookup query and the data stored in their biographical profiles.

In certain embodiments, the lookup query comprises one or more of: (i) a member identifier [e.g., text that identifies a member (e.g., a user-id or nickname included in a member biographical profile)], (ii) a member name, and (iii) searchable text (e.g., one or more keywords/keyphrases).

In certain embodiments, at step (d) the instructions cause the processor to: receive a user input corresponding to a selection of at least a portion of the one or more genetic profile matches for the user for addition to a list of followed members [e.g., via a user selection of one or more selectable buttons (e.g., “follow” buttons), each associated with a genetic profile match and displayed in the GUI]; add the selected genetic profile matches to the list of followed members, thereby identifying the selected genetic profile matches as followed members; and cause graphical rendering of, for presentation to the user, (e.g., within a user feed window of the GUI), graphics and/or text corresponding to a set of renderable data associated with a content object associated with (e.g., posted by) each of at least a portion of the followed members.

### Definitions

In order for the present disclosure to be more readily understood, certain terms used herein are defined below. Additional definitions for the following terms and other terms may be set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Genotyping data: As used herein, the term “genotyping data” refers to data obtained from measurements of a genotype. Measurements of a genotype performed on a biological sample identify the particular nucleotide(s) (also referred to as “bases”) that is/are incorporated at one or more particular positions in genetic material extracted from the biological sample. Accordingly, genotyping measurements for a particular individual are measurements performed on a biological sample of from the individual, and which identify the particular nucleotides present at one or more specific positions within their genome.

In certain embodiments, genotyping data describes an individual's phenotype. Genotyping data may be measurements of particular genes (e.g., portions of an individual's genetic sequence, e.g., DNA sequence), SNPs, or variants of SNPs. For example, a genotyping measurement of a particular SNP for an individual identifies the particular variant of that SNP that the individual has. A genotyping measurement of a particular gene for an individual identifies the particular nucleotides that are present at one or more locations within and/or in proximity to the gene for the individual. For example, genotyping measurements of a particular gene may identify the particular variants of one or more SNPs associated with a particular gene.

In certain embodiments, genotyping data is obtained from a multi-gene panel. In certain embodiments, genotyping data is obtained from assays (e.g., TaqMan™ assays) that detect one or more specific variants of specific SNPs. In certain embodiments, genotyping data is obtained from genetic sequencing measurements. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

Supplement: As user herein, the term “supplement” refers to a product ingested, consumed, and/or applied by a user in order to do at least one of: enhance wellbeing, improve performance or function, and counteract effects of a chronic condition. A supplement may be a vitamin, multivitamin, mineral, dietary supplement, herb, botanical, concentrate, metabolite, extract, amino acid, over-the-counter medication, prescription medication, topical product, or health/treatment regimen or program. In certain embodiments, a supplement is to be taken on a recurring basis (e.g., daily or twice daily) by a user for a period of time. A period of time may be an ongoing basis with no pre-determined cessation period. In certain embodiments, a supplement is a program or regimen that a user can enroll in or purchase access to. For example, a supplement may be a behavioral program such as a focus program or a personalized fitness plan (e.g., for use in home exercise).

Variant: As used herein, the terms “variant” refers to a specific variation of a specific SNP occurring in the genetic material of a population. In certain embodiments, a variant is a specific combination of a first allele of a first copy of an individual's genetic material (e.g. corresponding to an individual's paternal DNA) and a second allele of a second copy of an individual's genetic material (e.g. corresponding to an individual's maternal DNA), as occurs in diploid organisms (e.g. humans).

Qualifier: As used herein, the term “qualifier” refers to a classification (e.g. a label) of a particular variant of a given SNP. The qualifier associated with a given variant is the particular classification (e.g. label) of that variant. For example, a given variant may be associated with a particular qualifier of a predefined set of possible qualifiers. For example, a given variant may be associated with a qualifier selected from a group of labels such as “Adapt,” “Normal,” and “Gifted.” In certain embodiments, for a given variant of a given SNP, a qualifier corresponds to a classification of the given variant based on (i) the prevalence of the given variant within a population (e.g. if the variant is common, e.g. if the variant is rare) and/or (ii) a health-related trait associated with the variant. For example, a common variant may be associated with the qualifier “Normal”. A rare variant that confers a disadvantageous phenotype, such as a predisposition to high cholesterol, may be associated with the qualifier “Adapt” (e.g. classified as rare and disadvantageous). A rare variant that confers an advantageous phenotype, such as a predisposition to lower cholesterol, may be associated with the qualifier “Gifted” (e.g. accordingly, the variant is classified as rare and advantageous).

Variant object: As used herein, the term “variant object” refers to a data structure corresponding to (e.g. that is used to represent) a specific variant of a physical SNP and/or gene within a given genome (e.g., the genome of a human).

SNP object: As used herein, the term “SNP object” refers to a data structure corresponding to (e.g. that is used to represent) a specific single nucleotide polymorphism (SNP). In certain embodiments, a SNP object comprises a SNP reference that identifies the specific SNP to which the SNP object corresponds. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

Gene object: As used herein, the term “gene object” refers to a data structure corresponding to (e.g. that is used to represent) a specific physical gene within a given genome (e.g. the human genome).

Category: As used herein, the term “category” refers to a data structure corresponding to (e.g. that is used to represent) a particular health-related trait or characteristic.

Product, Genetic Profile Product, Personal Genetic Profile Product: As used herein, the terms “product,” “genetic profile product,” and “personal genetic profile product,” refer to a data structure corresponding to (e.g. that is used to represent) a general class of health-related traits and/or characteristics. In certain embodiments, a product is associated with one or more categories that correspond to health-related traits and characteristics related to the general class of health-related traits and characteristics to which the product corresponds.

Personal Genetic Profile Assessment: As used herein, the term “personal genetic profile assessment” refers to a data structure (e.g., a hierarchy of data structures) corresponding to (e.g. that is used to represent) the phenotype of a user for one or more general classes of health-related traits and/or characteristics. In certain embodiments, a personal genetic profile assessment of a user is generated by associating genotyping data of the user with premade (i.e., stored) generic personal genetic profile products. In certain embodiments, a user's personal genetic profile assessment is viewed using an assessment graphical user interface (“assessment GUI”) on a computing device (e.g., a smartphone).

Developer: As used herein, the term “developer” refers to a person, company, or organization that uses a graphical user interface to create data structures. In certain embodiments, a developer also genotypes a biological sample in response to an assessment corresponding to a product being purchased or made accessible to an individual.

User: As used herein, the term “user” refers to a person who uses an assessment graphical user interface in order to view information about a genome. The user may supply one or more biological samples to be genotyped in order for a personal genetic profile assessment to be formed. The user may purchase or be given access to one or more products in order to view a personal genetic profile assessment. The user may purchase one or more supplements from a list of purchase recommendations provided in the graphical user interface that are based on the user's personal genetic profile assessment. The terms “user” and “individual” are used interchangeably herein.

Graphical Control Element: As used herein, the term “graphical control element” refers to an element of a graphical user interface element that may be used to provide user and/or individual input. A graphical control element may be a textbox, dropdown list, radio button, data field, checkbox, button (e.g., selectable icon), list box, or slider.

Associate, Associated with: As used herein, the terms “associate,” and “associated with,” as in a first data structure is associated with a second data structure, refer to a computer representation of an association between two data structures or data elements that is stored electronically (e.g. in computer memory).

Provide: As used herein, the term “provide”, as in “providing data”, refers to a process for passing data in between different software applications, modules, systems, and/or databases. In certain embodiments, providing data comprises the execution of instructions by a process to transfer data in between software applications, or in between different modules of the same software application. In certain embodiments a software application may provide data to another application in the form of a file. In certain embodiments an application may provide data to another application on the same processor. In certain embodiments standard protocols may be used to provide data to applications on different resources. In certain embodiments a module in a software application may provide data to another module by passing arguments to that module.

### BRIEF DESCRIPTION OF THE DRAWINGS

Drawings are presented herein for illustration purposes, not for limitation. The foregoing and other objects, aspects, features, and advantages of the invention will become more apparent and may be better understood by referring to the following description taken in conjunction with the accompanying drawings, in which:

FIG. 1 is a block diagram illustrating associations between different data structures in personal genetic profile products, according to an illustrative embodiment of the invention;

FIG. 2 is a block diagram showing an organizational hierarchy of a personal genetic profile product, according to an illustrative embodiment of the invention;

FIG. 3A is a screenshot showing a homescreen of a graphical user interface (GUI) that a user uses to view different products that summarize their genetic profile, according to an illustrative embodiment;

FIG. 3B is a screenshot of the GUI of FIG. 3A showing the interface that appears when a particular product is selected, according to an illustrative embodiment;

FIG. 3C is a screenshot of the GUI showing a summary of the product that appears when the information (“i”) button of FIG. 3B is selected, according to an illustrative embodiment;

FIG. 3D is a screenshot of the GUI of FIG. 3A showing the interface that appears when a particular category of the selected product is selected, according to an illustrative embodiment;

FIG. 3E is a screenshot of the GUI of FIG. 3A showing the interface that appears when a particular SNP object of the selected category is selected, according to an illustrative embodiment;

FIG. 3F is a screenshot of the GUI showing further additional information that can be viewed by scrolling when the particular SNP object is selected, according to an illustrative embodiment;

FIG. 3G is a screenshot of the GUI showing further additional information that can be viewed by scrolling further when the particular SNP object is selected, according to an illustrative embodiment;

FIG. 3H is a screenshot of the GUI showing a summary of the category that appears when the information (“i”) button of FIG. 3D is selected, according to an illustrative embodiment;

FIG. 4 is a block diagram showing a process for creating a personal genetic profile assessment, according to an illustrative embodiment of the invention;

FIG. 5 is a portion of a text file comprising genotyping data, according to an illustrative embodiment of the invention;

FIG. 6 is a block diagram showing a process for automatically identifying, presenting, and matching a user to members of a dating pool based on genetic interests and/or shared genetic information via a graphical user interface, according to an illustrative embodiment;

FIG. 7 is a block diagram showing a process for automatically identifying and presenting to a user one or more nearby genetic profile matches based on shared genetic interests and/or similar (and/or compatible) genotypes via a graphical user interface, according to an illustrative embodiment;

FIG. 8 is a block diagram showing a process for automatically identifying and presenting to a user genetically tailored online content via a graphical user interface, according to an illustrative embodiment;

FIG. 9 is a block diagram showing a process for automatically managing and filtering social media interactions of a user with other social media platform members based on shared genetic interest and/or personal genetic data, according to an illustrative embodiment;

FIG. 10 is a block diagram of an example network environment for use in the methods and systems described herein, according to an illustrative embodiment of the invention; and

FIG. 11 is a block diagram of an example computing device and an example mobile computing device, for use in illustrative embodiments of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE INVENTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim. Documents are incorporated herein by reference as noted. Where there is any discrepancy in the meaning of a particular term, the meaning provided in the Definition section above is controlling.

Headers are provided for the convenience of the reader and are not intended to be limiting with respect to the claimed subject matter.

Presented herein are systems and methods for automatically managing and filtering social media interactions and/or online content based on permissioned personal genetic profiles of individuals. As described herein, such permissioned personal genetic profiles may be derived from genotyping data for the individual, for example from an individual's personal genetic profile assessment.

In particular, an individual's personal genetic profile assessment stores a collection of genotyping data for the individual, along with related information, in an organized fashion. In particular, an individual's personal genetic profile assessment includes data representing the results of one or more genotyping tests for the individual. Each genotyping test measures a set of SNPs to determine, for each SNP of the set, a particular variant of the SNP that the individual has.

SNPs correspond to specific locations within or nearby genes in an individual's genetic material (e.g. a SNP may occur in a promotor region that influences transcription of a particular gene, e.g. a SNP may occur within 5 kb upstream or downstream of a particular gene, e.g. a SNP may occur within 100 kb upstream or downstream of a particular gene, e.g. a SNP may occur within 500 kb upstream or downstream of a particular gene, e.g. a SNP may occur within 1 Mb upstream or downstream of a particular gene). Accordingly, the specific variant of a particular SNP that an individual has may influence the expression of one or more genes with which the SNP is associated (e.g., occurs within or nearby) which, in turn, influences various health related phenotypes for the individual. Accordingly, performing and supplying an individual with results of genotyping tests that determine the particular variants of a various SNPs that the individual has can provide the individual with insight into how their unique genetic makeup influences their unique physical and behavioral characteristics.

Different genotyping tests may measure different sets of SNPs. In certain embodiments, the different sets of SNPs measured in different genotyping tests are selected such that a particular genotyping test measures a particular set of SNPs that all are related to (e.g., influence physical and/or behavioral characteristics related to) a general class of health-related traits and characteristics. Accordingly, an individual may have one or more genotyping tests performed to gain insight into the different ways that their genetic makeup influences their health, physical characteristics, and behavior.

In addition to using their genotyping data (e.g., as stored via a personal genetic profile assessment) to gain insight into their genetic makeup, an individual may utilize their genotyping data along with the systems and methods described herein to obtain genetically tailored social media interactions and/or online content. To do so, an individual creates a permissioned personal genetic profile that is accessible by software systems responsible for managing, filtering, and presenting to them social media interactions and/or online content. The individual's permissioned personal genetic profile may comprise the same information as in their personal genetic profile assessment, or, as described herein, it may include a limited portion of the information in their personal genetic profile assessment and/or data created based on or derived from their genotyping data (e.g., as stored in their personal genetic profile assessment). Limiting the information content of their permissioned personal genetic profile may be useful for security and/or privacy considerations.

### Storage and Presentation of Personal Genetic Profile Assessments

### Flexible Data Structure Framework

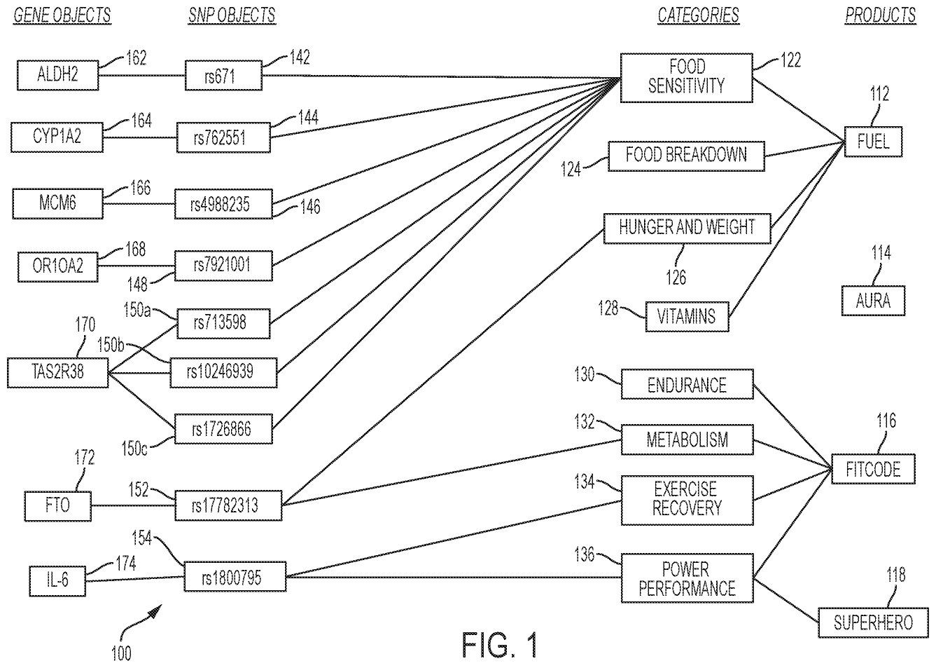

Turning to FIG. 1, in certain embodiments, in order to provide an individual not only with their personal genetic profile assessment, but also convey information related to the particular traits and characteristics that are influenced by the specific SNP variants present in their genetic material in an organized and intuitive fashion, the systems and methods described herein provide a framework comprising an intuitive hierarchical organization of data structures. The framework provides for storing relationships (e.g. associations) between particular SNPs, biological traits and characteristics, and general classes of such traits and characteristics, based on the specific traits that each particular SNP influences.

In certain embodiments, a first (e.g., top level) class of data structures, referred to herein as products, are used to represent different general classes of health-related traits and characteristics. In certain embodiments, a product data structure corresponds to a particular assessment ordered (e.g., purchased by the individual), in which unique versions of genes and/or SNPs that an individual has that influence the particular general class of health-related traits and characteristics that the corresponding product represents are identified (e.g., via genotyping measurements).

In certain embodiments, each product has a name (e.g. a product data structure comprises a name (e.g. text data representing the name)) that provides a convenient, and memorable way to refer to the product. For example, a particular product 112 (e.g. named “FUEL™”) is used to represent a class of traits corresponding to the way in which an individual's body processes different foods and nutrients. Another product 114 (e.g. named “AURA™”) is used to represent a class of traits corresponding to skin health. Another product 116 (e.g. named “FITCODE™”) is used to represent a class of traits corresponding to physical fitness. Another product 118 (e.g. named “SUPERHERO™”) is used to represent a class of traits corresponding to physical and intellectual performance. In certain embodiments, a name of a product is the same as the name under which a particular assessment is offered for sale. For example, assessments FUEL™, FITCODE™, AURA™, and SUPERHERO™ are offered for sale by Orgi3n, Inc. of Boston Mass.

In certain embodiments, each product is in turn associated with one or more of a second class of data structures, referred to as categories. In certain embodiments, each category corresponds to a particular health-related trait or characteristic (e.g. food sensitivity, food breakdown, hunger and weight, vitamins, skin uv sensitivity, endurance, metabolism, joint health, muscle strength, intelligence). In certain embodiments, the categories with which a particular product is associated each correspond to different health-related traits or characteristics that are related to the general class of health-related traits or characteristics to which the particular product corresponds (e.g. the general class of health-related traits or characteristics that the product represents). As with products, in certain embodiments, each category has a name (e.g. a category data structure comprises a name (e.g. text data representing the name)) that provides a convenient, and memorable way to refer to the category.

In turn, each category is associated with one or more SNP objects, each SNP object corresponding to a specific SNP. Each SNP object associated with a particular category corresponds to a specific SNP that influences a specific health related trait that relates to the trait or characteristic to which the particular category corresponds. Each SNP object may identify the specific SNP to which it corresponds via a SNP reference that the SNP object comprises. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

For example, the schematic of FIG. 1 shows an example of series of products, categories, and SNP objects that are associated with each other. Associated gene objects, to be described in the following, are also shown. The different products and categories are identified by their particular names, and the SNP objects each are identified by a respective SNP reference each comprises. In the example of FIG. 1, the SNP references are NCBI database reference numbers.

The “FUEL™” product 112 is associated with categories such as “Food Sensitivity” 122, “Food Breakdown” 124, “Hunger and Weight” 126, and “Vitamins” 128. Several SNP objects corresponding to specific SNPs that influence characteristics related to an individual's sensitivity to different types of foods, and, accordingly, are associated with the “Food Sensitivity” category 122 are shown. In FIG. 1, the lines connecting the SNP objects to different categories indicate the association of each particular SNP object with one or more different categories. The associations may be direct associations or indirect associations (i.e., through mutual association with an intermediate data structure not shown).

For example, SNP object 132 corresponds to the rs671 SNP, which influences the manner in which an individual processes alcohol. In particular, depending on the particular variant of the rs671 SNP that an individual has, the individual may process alcohol normally, or be impaired in their ability to process alcohol, and likely suffer from adverse effects resulting from alcohol consumption, such as flushing, headaches, fatigue, and sickness. Accordingly, providing individuals with knowledge of the particular variant of the rs671 SNP they possess may allow them to modify their behavior accordingly, for example, by being mindful of the amounts of alcohol that they consume (e.g. on a regular basis, e.g. in social settings).

Other SNP objects corresponding to SNPs that influence food sensitivity related characteristics, and, accordingly, are associated with the “Food Sensitivity” category 222 are shown. For example, SNP object 144 corresponds to the rs762551 SNP that influences caffeine metabolism, SNP object 146 corresponds to the rs4988235 SNP that influences lactose intolerance, and SNP object 148 corresponds to the rs72921001 SNP that influences an aversion to the herb cilantro (e.g. depending on the particular variant of this SNP that an individual has, they may either perceive cilantro as pleasant tasting or bitter and soap-like in taste).

In certain examples, multiple SNPs are associated with a particular characteristic and, accordingly, the SNP objects to which they correspond may be grouped together. For example, three SNPS—rs713598 (corresponding to SNP object 150a), rs10246939 (corresponding to SNP object 150b), and rs1726866 (corresponding to SNP object 150c),—influence the sensitivity of individuals to bitter tasting foods (e.g. cabbage, broccoli, cauliflower, kale, brussel sprouts, and collard greens), and, accordingly, their enjoyment of or aversion to such foods.

SNPs correspond to specific locations within or nearby (e.g., a SNP may occur in a promotor region that influences transcription of a particular gene, e.g., a SNP may occur within 5 kb upstream or downstream of a particular gene, e.g., a SNP may occur within 100 kb upstream or downstream of a particular gene, e.g., a SNP may occur within 500 kb upstream or downstream of a particular gene, e.g., a SNP may occur within 1 Mb upstream or downstream of a particular gene) genes in an individual's genetic material. Accordingly, in certain embodiments, as shown in FIG. 1, each SNP object is associated with a gene object that corresponds to the particular gene within or nearby to which the SNP to which the SNP object corresponds is present. For example, the rs671 SNP corresponds to a location within the ALDH2 gene; the rs762551 SNP corresponds to a location within the CYP1A2 gene, the rs4988235 SNP occurs within the MCM6 gene, and the rs72921001 SNP occurs within the OR10A2 gene. Accordingly, SNP object 142 (corresponding to the rs671 SNP) is associated with gene object 162 (corresponding to the ALDH2 gene). Similarly, SNP object 144 (corresponding to the rs762551 SNP) is associated with gene object 164 (corresponding to the CYP1A2 gene), SNP object 146 (corresponding to the rs4988235 SNP) is associated with gene object 166 (corresponding to the MCM6 gene) and SNP object 148 (corresponding to the rs72921001 SNP) is associated with gene object 168 (corresponding to the OR10A2 gene).

Other SNPs objects correspond to SNPs that are nearby particular genes of interest and thereby influence characteristics associated with expression of the gene. For example, rs12696304 is a SNP that lies 1.5 kb downstream from the TERC gene, and influences biological aging associated with the TERC gene. Accordingly, in one example, a SNP object corresponding to the rs12696304 SNP is associated a gene object corresponding to the TERC gene.

In certain embodiments, multiple SNPs of interest occur within a single gene. For example, the three SNPs related to bitter taste—rs713598, rs10246939, and rs1726866—occur within the TAS2R38 gene. Accordingly, SNP objects 150a, 150b, and 150c, which correspond to the rs713598, rs10246939, and rs1726866 SNPs, respectively, are all associated with a gene object 170 corresponding to the TAS2R38 gene.

In certain embodiments, different products correspond to different general classes of health-related traits and characteristics. For example, products may be based on particular organs (e.g. product 114, named “AURA™”, is related to skin health), or particular habits, activities, or bodily functions. For example, food related biological characteristics and traits may be covered by a single products or a plurality of products. A single product or a plurality of products may be based on learning and brain function characteristics and traits. A single product or a plurality of products may be based on physical fitness (e.g., cardiovascular strength, agility, flexibility, muscular strength).

For example, as shown in FIG. 1, another product 116 (e.g. named “FITCODE™”), relates to a general class of physical fitness related traits, and, accordingly, comprises categories associated with endurance 130 (“Endurance”), metabolism 132 (“Metabolism”), the ability of an individual to recover effectively following exercises 134 (“Exercise Recovery”), and cardiovascular fitness and skeletal muscle makeup 136 (“Power Performance”).

In certain embodiments, a particular SNP object is associated with two or more categories. For example, the rs17782313 SNP, occurring in the FTO gene, influences an individual's appetite. Accordingly, as shown in FIG. 1, the SNP object 152 corresponding to the rs17782313 SNP is associated with both the “Hunger and Weight” category 126 of the “FUEL™” product, and the “Metabolism” category 132 of the “FITCODE™” product. SNP object 152 is also associated with gene object 172, reflecting the fact that the rs17782313 SNP occurs in the FTO gene. In certain embodiments, as with the rs17782313 SNP object, each of a first category and a second category with which a particular SNP object is associated are associated with a different product. In certain embodiments, a particular SNP object is associated with a first category and a second category, and both the first category and the second category are associated with the same product.

For example, the SNP object 154 corresponding to the rs1800795 SNP of the IL-6 gene (accordingly, SNP object 154 is associated with gene object 174, which corresponds to the IL-6 gene) is associated with the “Exercise Recovery” category 134 and the “Power Performance” category 136, both of which are associated with the “FITCODE™” product 116. In addition, in certain embodiments, a category is associated with two or more products. For example, the “Power Performance” category 136 is associated with the “FITCODE™” product 116, as well as the “SUPERHERO™” product 118, which provides an assessment of a general class of traits related to physical and intellectual performance.

In certain embodiments the hierarchical organization of product, category, SNP object, gene object, and variant object data structures serves as a flexible template that facilitates both the rapid creation of individual personal genetic profile assessments from genotyping measurements taken from a plurality of individuals, and the presentation of an individual's personal genetic profile assessment. In particular, an individual may purchase assessments corresponding to different products, in order to gain insight into the manner in which their personal genome influences the different general classes of health-related traits and characteristics to which each different product corresponds. Accordingly, an individual's personal genetic profile assessment corresponding to one or more products comprises, for each specific SNP associated with each category that is associated with each of the one or more products, an identification of the particular variant of the specific SNP that the individual has. Typically, the identification is obtained via one or more genotyping measurements performed on a biological sample taken from the individual (e.g. a blood sample, e.g. a cheek swab sample, e.g. a saliva sample, e.g. a hair sample, e.g. hair follicle cells).

In certain embodiments, an individual may purchase a first assessment corresponding to a first product, and provide a biological sample for genotyping. The individual's biological sample may be stored (e.g. cryogenically frozen). After a period of time, the individual may choose to purchase additional assessments corresponding to other products, and the individual's previously stored biological sample may be taken from storage for additional genotyping measurements of the additional SNPs that are associated with the new products. Moreover, in certain embodiments, additional new products may be created over time, and new assessments corresponding to new products offered to and purchased by individuals. In certain embodiments, as new information related to the influence of new and/or existing SNPs on different specific health related characteristics is elucidated, new SNP objects and gene objects may be created, and new associations between them and new or existing categories and/or products established. In certain embodiments, existing personal genetic profile assessments of individuals are automatically updated to reflect new information.

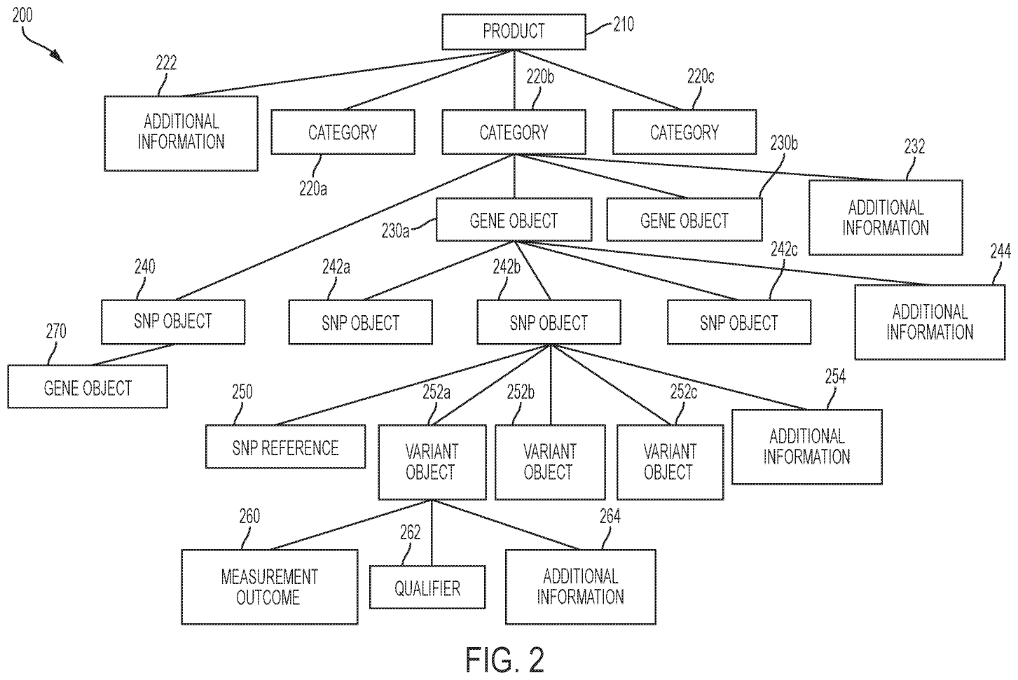

In certain embodiments, in order to facilitate the creation and presentation of individual personal genetic profile assessments (e.g. corresponding to one or more different products) based on the framework described above, the product, category, SNP object, and gene object data structures described herein are created and associated as a generic hierarchy of data structures to later be associated with the genotyping data of an individual. FIG. 2 is a block diagram of a hierarchy of data structures 200 of an example genetic profile product. In certain embodiments, a developer creates and stores one or more generic hierarchies of data structures in accordance with FIG. 2 that define one or more products that may be purchased and/or accessed by an individual. The hierarchies of data structures are generic in that they contain no personal information for any one individual, but instead define the collection of genes, SNPs, and variants that have relevance to the biological characteristics and/or traits that are encompassed by a product.

An exemplary data structure of each type is shown to be associated with sub-data structures in FIG. 2 in order to simplify presentation of the figure. It is understood that data structures may be associated to any number of other data structures in the hierarchy if the association is consistent with the associations shown in FIG. 2. For example, category 220b is shown to be associated with gene objects 230a-b while category 220c may be associated with one or more gene objects and/or SNP objects, but any such associations are not shown. In some embodiments, data structures may be created without also forming associations between other structures of relevant types. For example, unassociated or partially associated data structures may be created for planning purposes such as during product or category development (e.g., category 220a has no associations yet because its scope has not been determined yet by the user). For example, unassociated or partially associated data structures may be created to allow genotyping data to be associated with relevant gene objects or SNP objects in order to retain the data in a ready to use format in the event that the gene objects and/or SNP objects are later associated with one or more categories.

Referring now to FIG. 2, product 210 comprises three categories 220a-c and additional information 222. Additional information 222 may be a name of the product, an icon associated with the product, and/or a description of the product. Category 220b comprises two gene objects 230a-b, one SNP object 240, and additional information 232. Additional information 232 may comprise a name of the category, a background image associated with the category, an icon associated with the category, a category order identifier, and/or a description of the category. SNP object 240 is associated with gene object 270. Gene object 230a is associated to three SNP objects 242a-c. Categories may be associated directly to SNP objects, such as category 220b is associated with SNP object 240, or they may be associated indirectly such as SNP objects 242a-c are associated to category 220b via gene object 230a. The ability to form associations indirectly allows all SNP objects associated with a particular gene object to be associated with a category by forming a single association in cases where all SNP objects of a particular gene are relevant to a particular category. The ability to form associations directly allows a particular SNP object to be associated with a category without also forming an association with all other SNP objects associated with the gene object associated with the particular SNP object in cases where only one or a subset of SNP objects of a particular gene object are relevant to a category.

Gene object 230a is also associated with additional information 244. Additional information 244 may comprise one or more data structures comprising information such as a unique gene identifier that corresponds gene object 230a to a specific physical gene and descriptive information about the corresponding gene. The gene identifier may be an alphanumeric code such as an accepted name of the gene or other identifying mark or label capable of being stored electronically. Additional information may be stored as a single data structure or a plurality of data structures.

SNP object 242b is associated with SNP reference 250, and additional information 254. SNP reference 250 is a unique identifier of the SNP that corresponds the SNP object to a specific physical SNP. The SNP reference may be an alphanumeric code such as an accepted name of the gene or other identifying mark or label capable of being stored electronically. The SNP reference may be an alphanumeric code such as a National Center for Biotechnology Information (NCBI) database reference number. Additional information 254 may comprise one or more data structures with other descriptive information about the corresponding SNP.

Variants of a particular SNP can be represented within a corresponding SNP object using various combinations of data elements such as a measurement outcomes, and qualifiers. For example, a particular variant of a SNP can be identified by a measurement outcome, which is an identifier, such as an alphanumeric code, that identifies the specific alleles corresponding to the particular variant. For example, a measurement outcome such as the string “CC” identifies a first variant of the rs762551 SNP in which an individual has a cytosine (C) at the rs762551 position in each copy of their genetic material. A measurement outcome such as the string “AC” identifies a second variant of the rs762551 SNP in which an individual has a C in one copy and an adenine (A) in the other at the rs762551 position. A measurement outcome such as the string “AA” identifies a second variant of the rs762551 SNP in which an individual has an A at the rs762551 position in each copy of their genetic material.

A qualifier is an identifier, such as an alphanumeric code, that identifies a classification of a variant, wherein the classification may be based on the prevalence of the variant within a population, a health-related trait associated with the variant, and/or other relevant classification bases.

Qualifiers may be words or short phrases that characterize the variant. For example, “adapt” may be used to characterize variants that are uncommon and/or disadvantageous; “normal” may be used to characterize variants that are common and/or neither advantageous nor disadvantage; and “gifted” may be used to characterize variants that are uncommon and/or advantageous. Additional information may also be included within a SNP object to describe a particular variant.

In certain embodiments, measurement outcomes and qualifiers that identify and classify, respectively, the same variant are associated with each other to form a variant object associated with the SNP object. For example, variant object 252a comprises measurement outcome 260, qualifier 262. Variant object 252a is also comprises additional information 264. Additional information 264 comprises a description of the variant. For example, the additional information comprises a description of the specific health-related phenotype that an individual with the variant represented by variant object 252a exhibits or an explanation of the prevalence of the variant. A SNP object may be associated with a variant object to represent each variant of the particular SNP to which it corresponds. For example, SNP object is associated with three variant objects 252a-c.

In certain embodiments, the data structures described herein above are stored as a generic hierarchy for use in generating an individual's personal genetic profile assessment. A collection of data structures corresponding to genes, SNPs, and variants may be organized into one or more categories within a product (as visualized in FIG. 2, for example). Products can be personalized to a particular individual in order to provide them with specific information about their particular genome by populating or associating the generic product with the individual's genotyping data. In certain embodiments, a personal genetic profile assessment is used to populate an assessment graphical user interface (“assessment GUI”) through which an individual views an assessment of his/her genetic profile. In this way, the individual can view an assessment GUI that visualizes his/her personal genetic profile assessment by showing the individual the particular variants of SNPs that the individual has (e.g., organized in a hierarchy of products and categories).

### Presentation of Individual Personal Genetic Profile Assessments

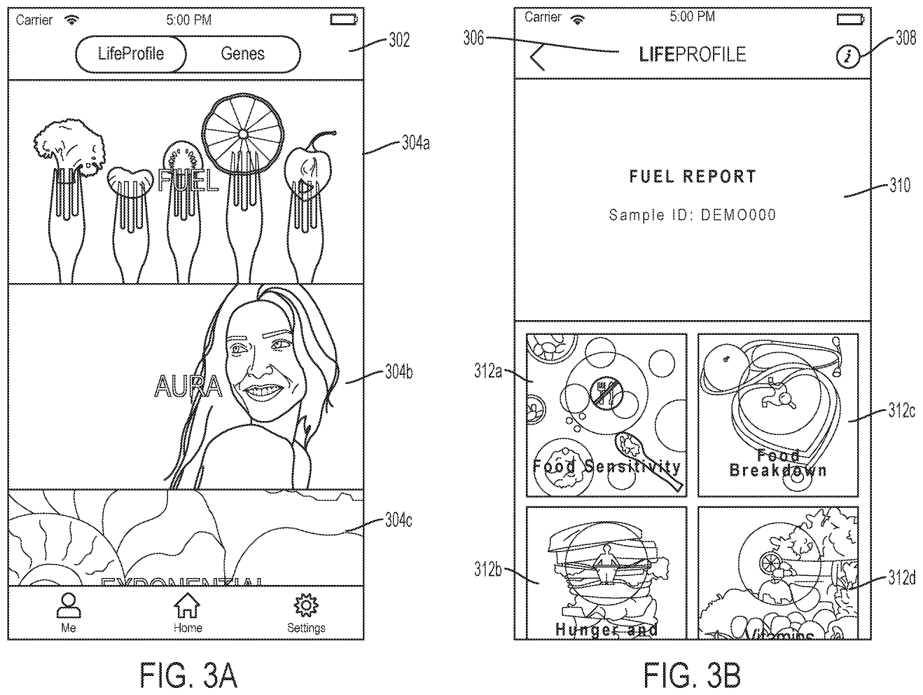

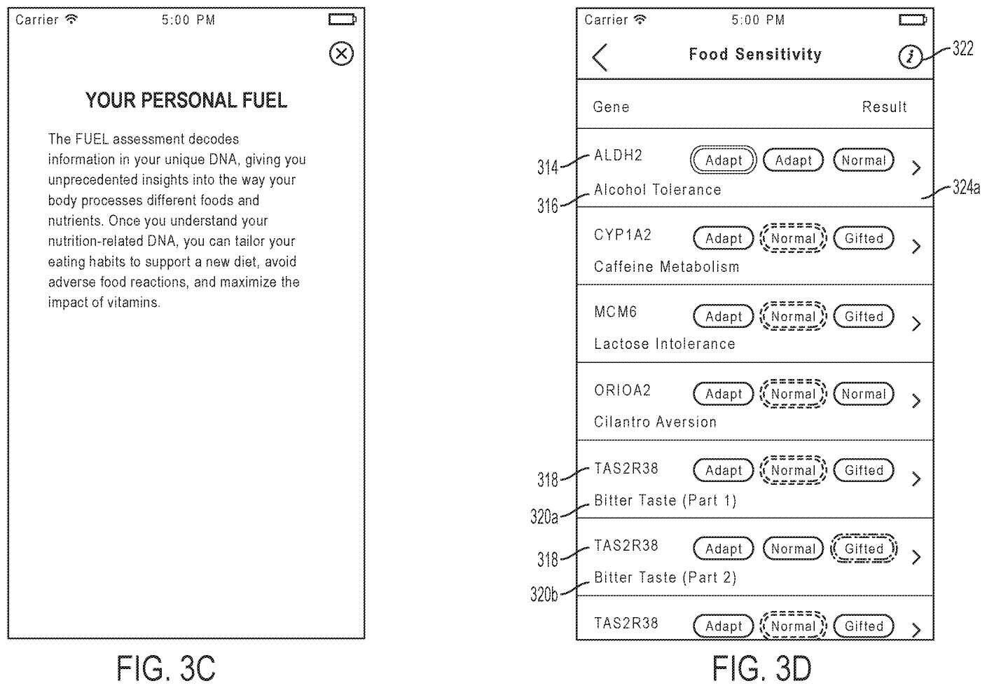

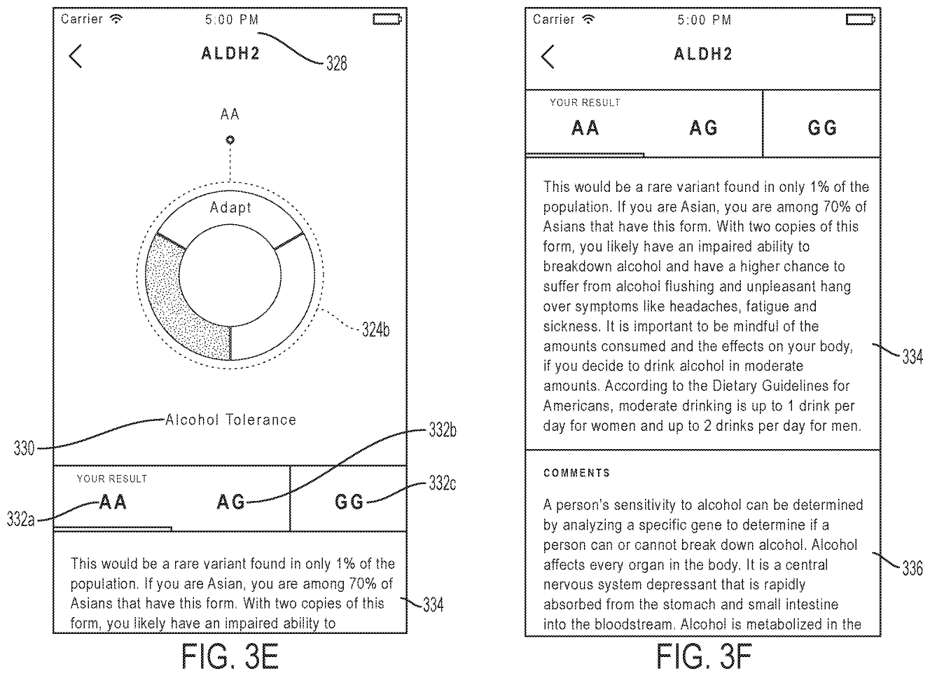

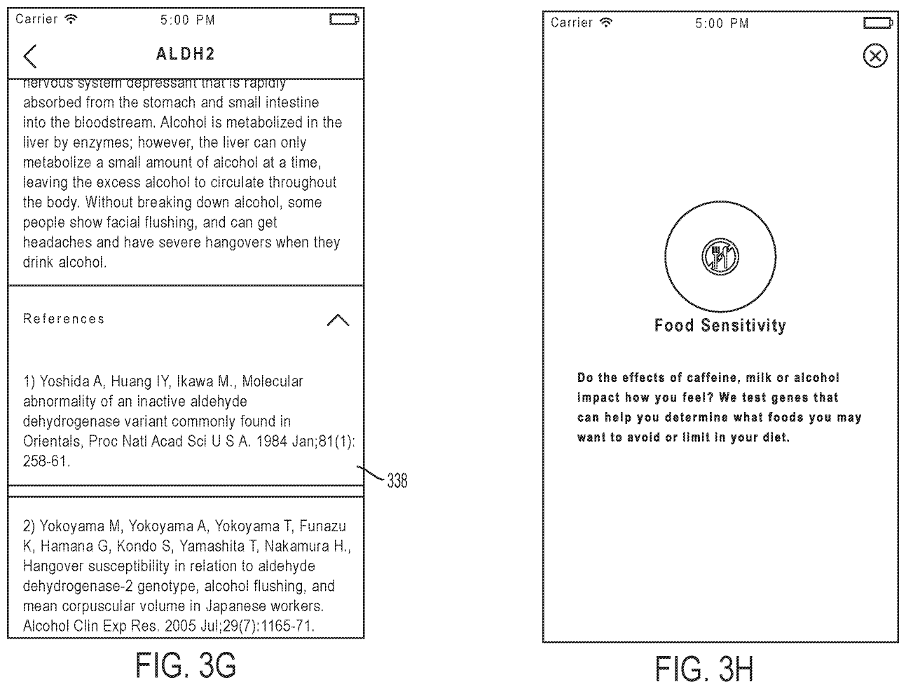

In certain embodiments, an individual views their genomic information using an assessment graphical user interface (assessment GUI) that is populated using one or more products (e.g., one or more hierarchies of data structures, such as the exemplary hierarchy of FIG. 2) and the individual's personal genetic profile assessment. In certain embodiments, the individual's personal genetic profile assessment is associated with the one or more products using a plurality of associations such that the assessment GUI is populated using the plurality of associations. In some embodiments, the one or more products are personalized by updating the hierarchies of data structures to comprise the data of the individual's personal genetic profile assessment such that the assessment GUI is populated using one or more products modified to be personalized to the individual. The assessment GUI allows individuals to interactively view their genomic information by navigating through the layers of data structures from the product level down to the level of information for individual SNPs. FIGS. 3A-3H are snapshots of an exemplary assessment graphical user interface that an individual would use to view their genomic information.

Referring now to FIG. 3A, the screenshot shows a home screen an individual uses to navigate to specific information about their personal genetic profile assessment. Three products 304a-c are visible: “FUEL™” 304a, “AURA™” 304b, “EXPONENTIAL™” 304c. Each product corresponds to a different set of genes that determine biological characteristics and traits. Selector 302 allows the individual to switch between his/her “LifeProfile™” that allows for navigation to specific information through the hierarchy of data structures, whereas Genes allows the individual to scroll through a listing of all SNPs corresponding to products that the individual has purchased or been given access to.

In certain embodiments, an assessment graphical user interface also includes a graphical control element for sharing data corresponding to an individual's personal genetic profile assessment (or a portion thereof) with one or more sharing entities. Sharing entities may be other individuals, people, or services with whom an individual wishes to share. For example, an individual may wish to share his or her entire personal genetic profile assessment with a friend, a spouse, or a social media service. Upon selection of the graphical control element for sharing, one or more graphical control elements may be provided for selecting which portions (e.g., products, categories, or a selected list of individual SNPs and/or genes) of a personal genetic profile assessment an individual would like to share (e.g., in the event that the individual would prefer certain portions of his or her personal genetic profile assessment remain private). In certain embodiments, the data is in a PDF report generated from the individual's personal genetic profile assessment.

Selecting a graphical control element for sharing may provide an individual with additional graphical control elements with which to select exactly with whom and by what method a personal genetic profile assessment (or portion thereof) is shared. For example, graphical control elements may be provided for selecting whether to text, email, or post the personal genetic profile assessment (or portion thereof) and other graphical control elements may be provided to allow an individual to select one or more recipients from among his contacts or enter contact information such as a phone number or email address. For example, an individual may select only certain friends or followers on a social media site with whom the personal genetic profile assessment (or portion thereof) is shared.

In certain embodiments, an individual uses a graphical control element for sharing in order to give access to other individuals using an assessment graphical user interface for viewing information about their genomes. For example, a first individual may use a graphical control element for sharing to give access to their personal genetic profile assessment to a spouse, wherein the spouse views the first individual's personal genetic profile assessment using an assessment GUI. In certain embodiments, an assessment GUI includes a graphical control element that an individual uses to select whose personal genetic profile assessment he or she is viewing. For example, the spouse may use such a graphical control element to toggle between viewing his or her own personal genetic profile assessment and the first individual's personal genetic profile assessment.

By selecting the “FUEL™” product from the LifeProfile™ listing of FIG. 3A, the individual sees the assessment GUI state of FIG. 3B. LifeProfile™ indicator 306 reminds the individual that he/she is using the LifeProfile™ navigation system. The information button 308 can be selected to view the brief description associated with the “FUEL™” product in its data hierarchy, as shown in FIG. 3C. Referring again to FIG. 3B, Fuel Report 310 provides space for a summary of the individual's “FUEL™” product genomic information. Categories 312a-d may be selected to view specific genomic information regarding different aspects of the individual's genome related to food and eating (e.g., different aspects of the “FUEL™” product). For each of the four categories, the individual sees the name of the category, the background image associated with the category, and the icon associated with the category. For example, category 312a is named Food Sensitivity, where the icon is a fork and knife with a slash, and the background image shows a variety of foods on a table.

Selecting the Food Sensitivity category 312a brings the individual to a view of the assessment GUI shown in FIG. 3D. The information button 322 can be selected to view the brief description associated with the Food Sensitivity category, as shown in FIG. 3H. Referring again to FIG. 3D, the individual may scroll through a list of selectable control elements corresponding to each of the SNPs related to the category, wherein each selectable control element comprises brief summary information that the individual may use to determine which selectable control element to select. For example, the first selectable control element in the list shown in FIG. 3D comprises a short description of a SNP 316, a graphical representation of a gene identifier 314 corresponding to the SNP, and a graphical representation of the qualifier 324a associated with the variant corresponding to the individual's particular alleles of the SNP.

The short description of the SNP 316 characterizes the biological characteristic or trait influenced by the corresponding SNP in the individual's genome. For example, the short description of the SNP 316 is “Alcohol Tolerance.” The individual would see that selecting the first selectable control element in the list would provide the individual with information about how the individual's genome influences his/her tolerance for alcohol consumption. The individual may select a particular selectable control element to view detailed information based on the short description of a SNP and/or the qualifier of the variant corresponding to his/her particular alleles of the SNP (as displayed by the graphical representation of the qualifier).

The graphical representation of the qualifier 324a is a graphic showing each of the qualifiers associated with the three variants corresponding to the SNP with the qualifier of the particular variant corresponding to the individual's alleles highlighted. Qualifiers may be words or short phrases that characterize the variant. For example, “adapt” may be used to characterize variants that are uncommon and/or disadvantageous; “normal” may be used to characterize variants that are common and/or neither advantageous nor disadvantage; and “gifted” may be used to characterize variants that are uncommon and/or advantageous. The graphical representation of the qualifier 324a highlights the qualifier associated with the variant corresponding to the individual's alleles in red. Different colors may be used to highlight different qualifiers in a graphical representation of a qualifier. For example, in FIG. 3D, when highlighted in a graphical representation of a qualifier, “adapt” qualifiers are highlighted in red, “normal” qualifiers are highlighted in blue, and “gifted” qualifiers are highlighted in green.

Some genes have multiple related SNPs. The related SNPs may influence a single biological characteristic or trait or a plurality of biological characteristics and/or traits. Each SNP may correspond to a unique selectable control element in the assessment GUI. For example, the graphical representation of the gene identifier 318 appears in two separate selectable control elements shown FIG. 3D since at least two unique SNPs relate to the gene corresponding to the graphical representation of the gene identifier 318. The two unique SNPs are differentiated by unique corresponding short descriptions 320a (“Bitter Taste (Part 1)”) and 320b (“Bitter Taste (Part 2)”). Short descriptions 320a and 320b correspond to related SNPs that influence an individual's sensitivity to bitterness in food.

Selecting the first selectable control element identified by short description 316 (“Alcohol Tolerance”) brings the individual to a view of the assessment GUI shown in FIG. 3H comprising detailed information regarding the SNP corresponding to the short description “Alcohol Tolerance.” The graphical representation of the gene identifier 328 is shown at the top of the screen. A graphical representation of the qualifier 324b associated with the variant corresponding to the individual's particular alleles of the SNP identified in the first selectable control element. Graphical representation 324b displays both that the measurement outcome corresponding to the individual's alleles is “AA” and that the qualifier associated with this variant is “Adapt”. The other two segments of the ring in graphical representation 324b relate to the other two variants corresponding to the SNP and are color coded to the associated qualifiers as described above. The graphical representation 324b is an alternative to the graphical representation 324a of FIG. 3D. Graphical control elements 332a-c indicate the measurement outcomes associated with each of the three variants corresponding to the SNP. Graphical control element 332a indicates that the individual's alleles correspond to the variant identified by the displayed measurement outcome (by displaying “Your Result” above the measurement outcome) as well as that information currently displayed below the row of graphical control elements 332a-c is associated with that variant (by displaying the light blue bar under the measurement outcome). A portion of description 334 associated with the variant identified in graphical representation 332a is visible. An individual may select other graphical control elements identified by other measurement outcomes to view information associated with other variants.

Referring now to FIG. 3F, the individual may scroll in order to read more information regarding their genome. By scrolling, the complete description 334 may be read as may other additional information 336, which may be include a brief description associated with a SNP object corresponding to the SNP. Scrolling further, an individual can see references 338 that provide further detail related to the currently selected variant of the SNP object, as shown in FIG. 3G.

The assessment GUI shown in FIGS. 3A-3H is configured for display on mobile devices (e.g., smartphones, tablets, PDAs), but an assessment GUI may also be configured for viewing on a computing device using the web (e.g., with a laptop or desktop computer). The assessment GUI is populated using data associated with one or more products. A standardized graphical user interface element (e.g., widget) is used to create data and data structures as well as associations between existing and new data and data structures.

### Automated Creation of Individual Personal Genetic Profile Assessments

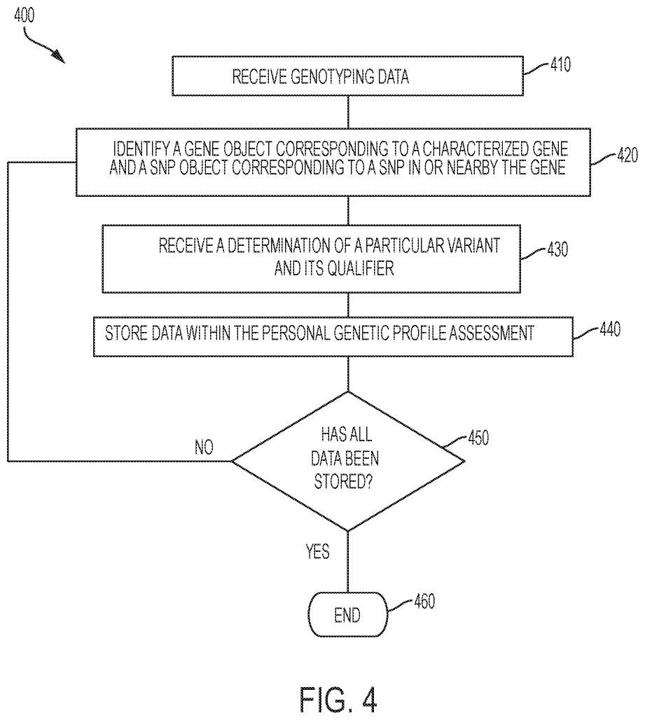

In certain embodiments, in order to populate an assessment GUI to provide to an individual, genotyping data must be added or associated to the individual's personal genetic profile assessment. FIG. 4 is a block diagram of exemplary method 400 for adding genotyping data to an individual's personal genetic profile assessment. In step 410, a processor of a computing device receives genotyping data. In step 420, the processor identifies a gene object corresponding to a gene measured in the genotyping data and a SNP object corresponding to a SNP in or nearby the gene (e.g. the SNP occurring within the gene or occurring nearby the gene (e.g. within a promotor region that influences transcription of the gene, e.g. within 5 kb upstream or downstream of the gene, e.g. within 100 kb upstream or downstream of the gene, e.g. within 500 kb upstream or downstream of the gene, e.g. within 1 Mb upstream or downstream of the gene). In certain embodiments, genotyping data is stored as a table of data in a text file where each row corresponds to a unique SNP. In step 430, a particular variant of the identified SNP object and its associated qualifier are determined based on data from genotyping measurements. For example, data corresponding to the measurement outcome of a particular variant may be stored as one or more columns at the end of each row. In step 440, the data is stored in the individual's personal genetic profile assessment. In accordance with method 400, at step 440, the data may be stored in a (previously generic) hierarchy of data structures or the data may be stored separately along with an association between the data and the identified gene object and SNP object. In any case, the stored data (and any generated and stored associations) define the personal genetic profile assessment for the individual. In step 450, the processor determines if all data of the genotyping data has been stored. If all data has not been stored in the individual's personal genetic profile assessment, then the method returns to step 420. If all data has been stored, then the method ends 460. In some embodiments, the processor determines if unstored data exists by determining if there is a row of data in the genotyping data below the just processed row.

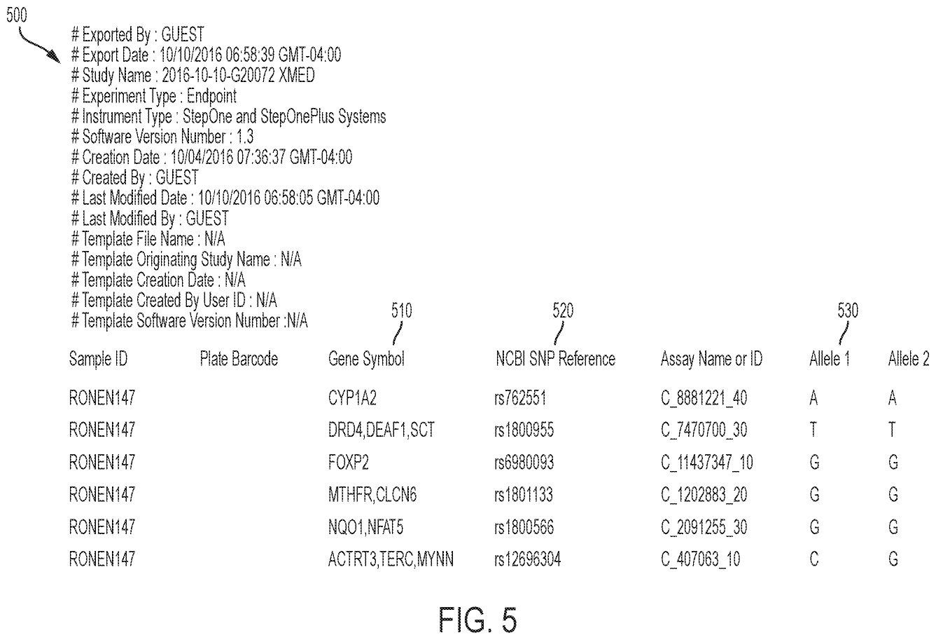

FIG. 5 shows exemplary genotyping data 500 that may be added to an individual's personal genetic profile assessment in accordance with method 400. Genotyping data may take the form of a text file saved by a user, wherein the text file is generated manually or as output from equipment for performing genotyping measurements (e.g. TaqMan™ SNP genotyping assays). FIG. 5 comprises 6 rows of genotyping data from a single biological sample (“RONEN147”). Each row corresponds to data for a different SNP. Each SNP of genotyping data 500 is identified by at least a gene identifier 510 and a SNP reference 520. The gene identifier identifies the gene with which the SNP is associated. In certain embodiments, multiple (e.g. two or more) genes are associated with the SNP (e.g. the SNP may occur nearby two or more genes and influence phenotypes associated with each of the associated genes), and, accordingly, two or more corresponding gene identifiers are listed. Each SNP in the genotyping data has a corresponding variant identified by the allele measurements 530. The measurements “allele 1” and “allele 2” for a given SNP may be compared with measurement outcomes associated with the variants of a SNP object corresponding to the given SNP to populate an individual's personal genetic profile assessment.

The genotyping data in FIG. 5 used to populate an individual's personal genetic profile assessment is generated from one or more biological samples of the individual. However, the one or more biological samples used in populating an individual's personal genetic profile assessment may also be taken from a different human or a non-human animal. In some embodiments, genotyping data is generated from one or more biological samples of a non-human animal. For example, an individual may supply biological samples of his or her pet in order to understand information about the pet's phenotype in order to assist in providing better care. The animal may be a pet or may be an animal cared for by an individual. For example, the individual may be a veterinarian or a caretaker at a zoo charged with caring for the animal. In some embodiments, genotyping data is generated from one or more biological samples of a ward to whom the individual is a guardian. For example, a parent may supply one or more biological samples to genotyping data for their child in order to improve his/her childrearing.

### Permissioned Personal Genetic Profiles and Accessible Genetic Profile Objects

In certain embodiments, a permissioned personal genetic profile is created for a user to provide access to identifications of genetic interests for the user and/or specific portions of their genotyping data (e.g., their personal genetic profile assessment). A user's permissioned personal genetic profile may use a variety of data structures to represent genetic interests and/or genetic information. In particular, a user's permissioned personal genetic profile may comprise various genetic interest identifiers, accessible variant objects, secondary profile tags, and combinations thereof.

### Genetic Interest Identifiers

Genetic interest identifiers correspond to various genomic objects (e.g., products, categories, gene objects, SNP objects, variant objects as described herein) and serve to identify particular genetic traits as well as individual SNPs and/or genes, sets of SNPs and the genetic traits they influence, and even specific variants of SNPs and/or genes that are of interest to the user. A genetic interest identifier may do so by identifying a stored genomic object, such as a particular product, category, gene object, SNP object, or variant object through a textual label that can be matched to the stored genomic object. For example, the textual label “Food Sensitivity” may be stored as a genetic interest identifier to identify the category “Food Sensitivity” 122 shown in FIG. 1. Textual labels that can be matched with any of a product name, category name, gene identifier, SNP reference may be used to identify particular products, categories, genes, and SNPs, respectively. Combinations of textual labels may be used to identify particular variant objects that represent specific variants of specific SNPs. For example, genetic interest identifier may combines a textual label that can be matched with a SNP reference with one or more textual label(s) that can be matched to a measurement outcome and/or qualifier for the specific SNP that the SNP reference identifies.

Genetic interest identifiers may be included in a user's permissioned personal genetic profile as a convenient way for them to identify interests based on their genotype without necessarily limiting their interests to their specific genotype and/or divulging their particular genotype information. For example, a user who has a genetic predisposition to having difficulty building lean muscle, but who is interested in increasing their muscle mass may include a genetic interest identifier corresponding to the “Power Performance” category 136. The user may then be matched with content that provides them with information (e.g., social media posts, online media, etc.) that allows them to exercise more efficiently to overcome their genetic predisposition. The user may also be matched with, for example, nearby genetic profile matches who have also expressed a similar interest (e.g., via inclusion of the same and/or related genetic interest identifiers in their permissioned personal genetic profiles) and/or granted access to genetic information related to this category (e.g., via inclusion of variant objects representing variants of SNPs within this category; e.g., via inclusion of related secondary profile tags (e.g., physical fitness tags)). In this manner, the user may be matched not only with those of similar genotypes, but also with those having complementary genotypes, for example, those with a genetic predisposition to building lean muscle.

### Accessible Variant Objects

In certain embodiments, a user's permissioned personal genetic profile includes one or more accessible variant objects, each representing a particular variant of a particular SNP that the user has and to which they have granted access. As described herein, variant objects may identify and/or classify specific variants of SNPs via measurement outcomes and/or qualifiers, respectively. An accessible variant object in a user's permissioned personal genetic profile may include one or both of a measurement outcome and a qualifier. Accordingly, by including various accessible variant objects in their permissioned personal genetic profile, a user may select which portions of their genotype to permit access to.

### Secondary Profile Tags

In certain embodiments, a user's permissioned personal genetic profile includes one or more secondary profile tags. Secondary profile tags are labels (e.g., textual labels) that represent information that can be determined from a user's genotyping data and used to build a secondary profile that relates to a particular activity, characteristic, or set of interests for the user.

For example, a dietary profile comprising one or more user-specific dietary tags may be determined for a user based on their genotyping data. The dietary tags identify common diets and/or allergens and provide a way to represent dietary guidelines and/or taste preferences for the user. For example, dietary tags such “vegetarian”, “vegan”, “pescatarian”, “low-cholesterol”, “dairy-free”, “lactose-free”, “gluten-free”, “paleo”, “low-sugar”, and the like may be used to identify various diets that, based on the user genotyping data, are recommended. For example, dietary tags such as “dairy”, “peanut”, “nut”, “gluten”, and the like, may be used to identify allergens that the user's genotyping data results indicates that they are allergic to. The dietary tags may be determined from the user genotyping data based on their association with particular variants of various different SNPs and/or qualifiers that classify them.

For example, SNPs associated with the FADS1, KCTDIO and PPARg genes influence cholesterol and fat storage levels. Accordingly, based on the presence of a variant and/or qualifier for any SNPs associated with these genes in a user's genotyping data, tags such as “low-cholesterol” may be determined for the user.

In certain embodiments, a physical fitness profile, similar to the above described dietary profile, may be determined for the user based on their genotyping data. The physical fitness profile may comprise a set of user-specific fitness tags that identify specific workout classifications (e.g., that are recommended for the user; e.g., that the user should avoid) (e.g., alphanumeric strings such as “HIIT”, “aerobic”; “cardio”; “high intensity”, “flexibility”, and the like) having been determined, by the processor, as associated with (e.g., beneficial to) the user based on their genotyping data.

Accordingly, in certain embodiments, the systems and methods described herein determine various secondary profile tags, such as dietary tags, fitness tags, etc., for the user. The user may include all determined secondary profile tags, or specific secondary profile tags in their permissioned personal genetic profile. In certain embodiments, this allows the user to not only generalize genotyping data in a useful fashion, but also add a layer of obfuscation between their permissioned personal genetic profile and their specific genotyping data.

### Creating and Updating Permissioned Personal Genetic Profiles

A user may initially create, and then update their permissioned personal genetic profile based on, for example, changes in their interest and as new genetic information becomes available—for example if they order new genotyping tests. In certain embodiments, the systems and methods described herein allow a user to build and/or update their permissioned personal genetic profile via profile builder graphical user interface (GUI). The profile builder GUI allows a user to select particular accessible genetic profile objects (e.g., genetic interest identifiers; e.g., accessible variant objects; e.g., secondary profile tags) that they wish to include or exclude from their permissioned personal genetic profile.

For example, the profile builder GUI may include selectable graphics and/or text that allow a user to select particular accessible genetic profile objects for inclusion and/or exclusion from their permissioned personal genetic profile. For example, the profile builder GUI may allow a user to select particular products, categories, gene objects, SNP objects, and variant objects (including measurement outcomes and/or qualifiers) to store as genetic interest identifiers in their permissioned personal genetic profile. The profile builder GUI may also allow the user to select SNPs and/or genes for which they have genotyping measurements and then store accessible variant objects to represent the particular SNP and/or gene variants that the user has in their permissioned personal genetic profile.

In certain embodiments, the systems and methods described herein automatically determine an initial set of secondary profile tags for the user, and present them to the user for selection, for example via presentation of selectable graphics and/or text for each secondary profile tag. The user may then select a subset of the initial set of secondary profile tags for inclusion in their permissioned personal genetic profile assessment.

The profile builder GUI may also allow a user to select particular accessible genetic profile objects to exclude from their genetic profile. In certain embodiments, the profile builder GUI comprises, for each of one or more accessible genetic profile objects stored in the permissioned personal genetic profile of the user, selectable graphics and/or text identifying the accessible genetic profile object. In this manner, the user may select particular accessible genetic profile objects to exclude from their permissioned personal genetic profile. The accessible genetic profile objects selected for exclusion from the user's permissioned personal genetic profile may then be removed from their permissioned personal genetic profile.

In certain embodiments, the user may also provide (e.g., as input via the profile builder GUI) a weighting value for each of one or more genetic profile objects. The user specified weighting values indicate a level of desired similarity and/or dissimilarity and/or compatibility in permissioned personal genetic profiles of matching individuals and/or genetic profiles of content objects. Examples of weighting values include, without limitation, binary values that can be used to label genetic profile objects as important or not (to the user), or desired similarity versus desired dissimilarity, as well as values along a sliding scale with one extrema corresponding to highly desired dissimilarity and the other corresponding to highly desired similarity.

In certain embodiments, the profile builder GUI may present selectable graphics and/or text representing various accessible genetic profile objects for an individual to select for inclusion and/or exclusion in their permissioned personal genetic profile in accordance with the hierarchical organization of products, categories, gene objects, SNP objects, and variant objects described herein. For example, the profile builder GUI may allow a user to select various accessible genetic profile objects representing specific genetic interests and/or variants by moving through multiple views of the GUI in a similar fashion to the manner in which the individual can view their personal genetic profile assessment using the assessment GUI described herein.

The profile builder GUI may be provided to the user as a stand-alone application, part of a personal genetic profile assessment GUI, or as a plug-in that is initiated from another application, such as one that provides genetically tailored matching and/or content delivery in accordance with the systems and methods described herein.

### Genetically Tailored Social Media Matching and Delivery of Online Content

Accordingly, by virtue of the permissioned personal genetic profiles described herein, the systems and methods described herein provide for matching of individuals and/or content delivery based on their genetic interests and/or portions of their genotypes that they have granted access to.

Potential Matches from a Dating Pool.

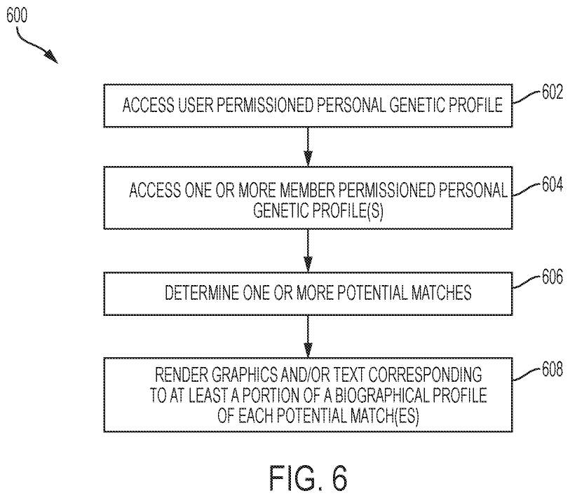

Turning to FIG. 6, in certain embodiments, the systems and methods described herein provide for automated identification of, presentation of, and matching a user to members of a dating pool based on genetic interests and/or shared genetic information. FIG. 6 shows an example process 600 for identifying potential matches from a dating pool and presenting them to the user via a GUI. In one step 602, the user's permissioned personal genetic profile is accessed. In another step 604, permissioned personal genetic profiles of one or more members of the dating pool are accessed. In another step 606, the various dating pool members' permissioned personal genetic profiles are evaluated in comparison with the user's permissioned personal genetic profile and one or more of the members are determined to be potential matches based the comparison between their permissioned personal genetic profiles and that of the user.

A variety of approaches may be used to determine potential matches. For example, a binary classification, determined based on a computed formula or output from a machine learning module (e.g., that takes as input the permissioned personal genetic profiles of the user and a particular member being evaluated) may be used to classify members as potential matches or not. In certain embodiments, for each of one or more members of the dating pool, a compatibility index is computed for the member and the user, based on their permissioned personal genetic profiles. The compatibility index may be used in a thresholding approach to classify various members as potential matches or not, and, in certain embodiments, to rank levels of compatibility. The compatibility index itself may be computed using a machine learning module (e.g., that takes as input the permissioned personal genetic profiles of the user and a particular member being evaluated).

In certain embodiments, information in biographical profiles of the user and the members is also taken into account.

Notably, identifying potential matches in this manner does not correspond to merely identifying genetically similar individuals, but can include taking into account desirable dissimilarities in genetic information between the user and their potential matches. For example, an individual with a genetic predisposition towards risk aversion may benefit from, and enjoy a relationship with another who has a genetic predisposition to risk seeking behavior, and vice versa. In certain embodiments, dissimilarities may be intentionally desired and are represented in user and member permissioned personal genetic profiles via weighting values that reflect a desired dissimilarity. In certain embodiments, beneficial dissimilarities and/or compatible genotypes are automatically identified, for example via a machine learning module, and/or are known a-priori and included in the computation of the compatibility index.

In certain embodiments, once one or more potential matches are identified, in another step 608, graphics and/or text corresponding to at least a portion of a biographical profile of each potential match are rendered for presentation to the user, for example via a dating app GUI. The rendered portion of the biographical profile may include one or more pictures of the potential match, as well as text describing their various interests and/or a short personal statement.

In certain embodiments, once a potential match is identified and presented to the user, the user may input an interest indicator to confirm their interest in the potential match. The interest indicator may have binary values (e.g., representing interested or not interested), or may have additional gradations of positive interest (e.g., representing interested and very interested). Likewise, the user themselves may be presented to the potential match to allow the potential match to confirm their interest in the user. If both the user and the potential match have input an interest indicator having an affirmative (e.g., positive interest) value with respect to each other, the potential match is determined to be a positive match. The user may then be presented with a notification of positive matching.

Accordingly, in this manner, the systems and methods described herein allow for a large dating pool can be narrowed down and presented to a user based on genetic interest and/or compatible genetic information between the user and various members of the dating pool.

### Nearby Genetic Profile Matches

In certain embodiments, the systems and methods described herein provide for permissioned genetic profile based matching of a user to nearby members of a genetic profile matching service. As described herein, identifying nearby genetic profile matches allows a user to, for example, coordinate their activities with other members of the genetic profile matching service based on particular traits such as dietary needs, fitness characteristics, and the like. Accordingly, by combining (i) location data (e.g., GPS data) for the user and members of the genetic profile matching service with (ii) permissioned personal genetic profiles of the user and members, the approaches described herein allow a user to identify nearby genetic profile matches.

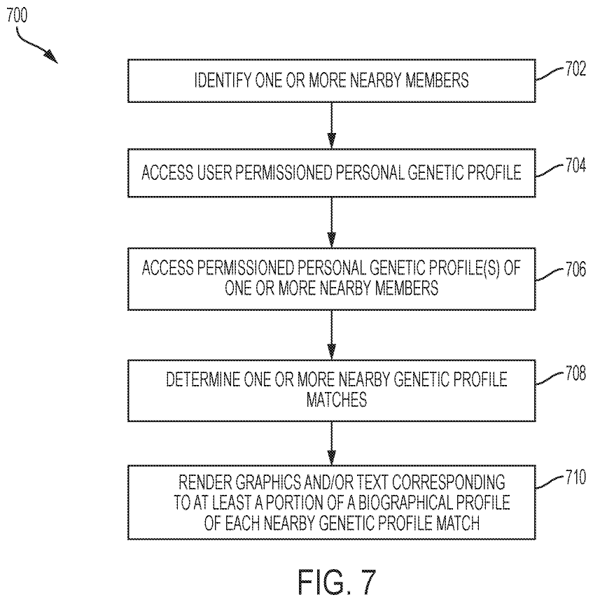

FIG. 7 shows an example process 700 for identifying nearby genetic profile matches. In one step 702, one or more members of a genetic profile based matching service that are in geographic proximity (e.g., nearby to) the user are identified. Nearby members may be identified using location data for the user and each of the one or more members. Location data may include GPS data provided, for example, by smartphones of the user and other members of the genetic profile based matching service. Location data may also include other data, such as check-ins/registrations at various locations, such as particular merchants, gyms, venues (e.g., for conferences, concerts, and other various gatherings), and the like.

In another step 704, the user's permissioned personal genetic profile is accessed. In another step 706, permissioned personal genetic profiles of each of the one or more nearby members are accessed. In another step 708, the permissioned personal genetic profile of the user is compared with those of the nearby members to identify one or more nearby genetic profile matches.

Various approaches, similar to those described herein with regard to determining potential matches from a dating pool, may be used to match the user to one or more nearby members based on their respective permissioned personal genetic profiles. In particular, binary classifications may be performed (e.g., genetic profile match or not) and/or a compatibility index determined for each member based on the member's permissioned personal genetic profile and that of the user.

In another step 710, graphics and/or text corresponding to at least a portion of a biographical profile for each nearby match are rendered for presentation to the user (e.g., via a GUI). The graphics and/or text may be rendered in a location dependent fashion, so as to convey to the user the location of each of at least a portion of the nearby genetic profile matches with respect to the user's own location. For example, a small picture of each nearby genetic profile match may be displayed on a map at a position corresponding to their actual location (e.g., as determined by their location data).

### Genetically Tailored Online Content

In certain embodiments, the systems and methods described herein facilitate providing a user with genetically relevant online content in general. In particular, online content may be represented by various content objects that can in turn be associated with particular genetic profiles.

Content objects may include the biographical profiles of dating pool members and genetic profile based matching services described herein. The genetic profiles associated with such content objects are, as described herein, the permissioned personal genetic profiles of particular member that a particular biographical profile represents.

Content objects may also be member generated content, such as online member posts to forums such as social media platforms, message boards, and the like. Such content objects may include renderable text and/or graphics created or curated (e.g., linked to; e.g., embedded within the content object) by the member. Member post content objects can be associated with permissioned personal genetic profiles of the members that created them. A user may then grant a particular forum (e.g., social media platform, message board, and the like) access to their permissioned personal genetic profile in order to be matched with genetically relevant content objects. In particular, such matching may be accomplished based on, for each member post, the permissioned personal genetic profile of the member that created the post and that of the user. A variety of approaches, analogous to those described above with regard to matching a user to potential matches and/or nearby genetic profile matches, may be used to perform such matching based on permissioned personal genetic profiles.

In certain embodiments, content objects are not limited to posts generated by individual members, but may also include commercial postings (e.g., sponsored posts) generated by corporations (e.g., as advertisements), media services, and the like, as part of commercial activity. Such commercial postings, though not necessarily associated with a particular member of the online forum, may nonetheless be associated with a genetic profile. In particular, commercial postings may be associated with content-specific genetic profiles that use accessible genetic profile objects to identify genetic interests for which the content of the commercial posting is relevant and/or genetic information (e.g., identifications of particular variants of particular SNPs) that identifies a subset of genotypes for which the commercial posting is relevant. Accordingly, the accessible genetic profile objects that content-specific genetic profiles comprise are the same as those used to created permissioned personal genetic profiles for various individuals. In certain embodiments, the content-specific genetic profile of a content object is created by a developer (e.g., via a GUI, similar to the profile builder GUI described herein). In certain embodiments, the content-specific genetic profile is generated automatically, for example by applying natural language processing to text included in the content object to automatically generate a series of genetic profile objects based on the text that the content object comprises.

Accordingly, in certain embodiments, the systems and methods described herein provide for filtering and automated aggregation and delivery to a user of genetically relevant content from online forums.

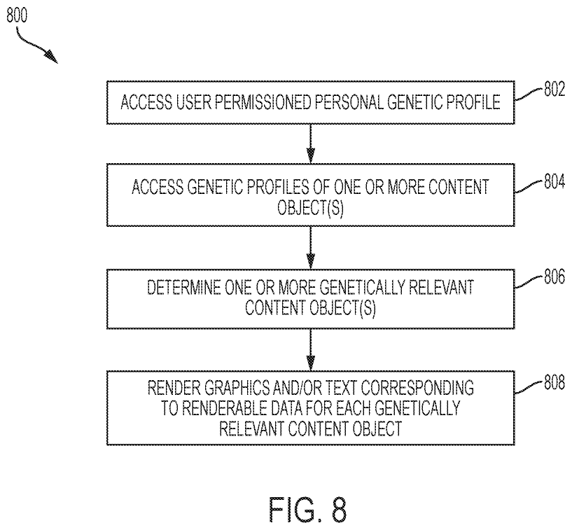

Turning to FIG. 8, a user may be matched to genetically relevant content objects via example process 800. In one step 802, a user's permissioned personal genetic profile is accessed. In another step 804, genetic profiles of one or content objects are accessed. In another step 806 one or more genetically relevant content objects are matched to the user based on, for a given content object, a genetic profile (e.g., a member permissioned personal genetic profile; e.g., a content-specific genetic profile) associated with the content object and the permissioned personal genetic profile of the user.

In another step 808, graphics and/or text corresponding to renderable data for each genetically relevant content object are rendered for presentation to the user. In this manner, the systems and method described herein allow for automated self-aggregation of online forums, and delivery to a user of genetically relevant online content.

For example, a user's feed on a social media platform typically comprises various postings by different members of the social media platform. Accordingly, in certain embodiments, the particular postings that appear and/or the order in which they appear, can be tailored for a particular user based on the user's permissioned personal genetic profile and the permissioned personal genetic profiles of the members who generated the member postings. For example, social media members may post content related to workouts and/or food they have eaten. Content posted by members having similar dietary restrictions and/or fitness needs (e.g., propensity to particular types of injuries, e.g., based on SNPs that influence joint health) to a particular user may, accordingly, be prioritized for display on the user's social media feed to provide the user with information on workouts and/or foods that may be beneficial to them.

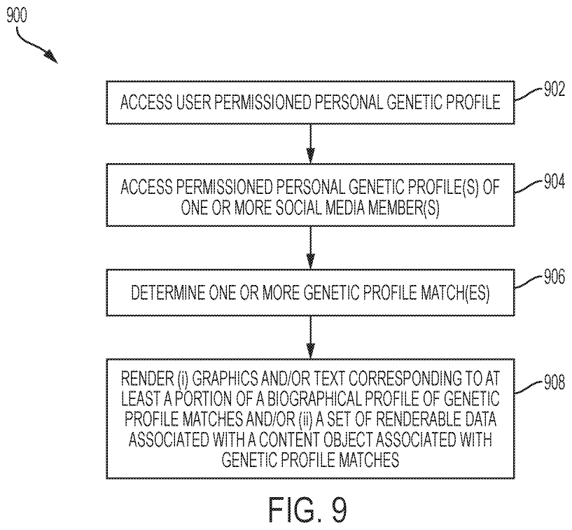

FIG. 9 shows an example process 900 for managing a user's social media feed in this manner. In one step 902, the user's permissioned personal genetic profile is accessed. In another step 904, permissioned personal genetic profiles of one or more members of the social media platform are accessed. In another step 906, certain social media platform members are determined to be genetic profile matches to the user based on their permissioned personal genetic profiles and that of the user. In another step 908, graphics and/or text corresponding to (i) at least a portion of a biographical profile of each of the one or more genetic profile matches for the user and/or (ii) a set of renderable data associated with a content object associated (e.g., posted by) each of at least a portion of the one or more genetic profile matches for the user are rendered for presentation to the user. The rendered graphics and/or text may be displayed in a social media GUI for viewing by the user.

In certain embodiments, graphics identifying genetic interests and/or traits that the user has in common with a genetic profile match are displayed in conjunction with the renderable data associated with a content object associated (e.g., posted by) with the genetic profile match. Common genetic interests and/or traits between the user and a genetic profile match to be displayed may be identified based on a matching between (i) accessible genetic profile objects of the permissioned personal genetic profile of the user and (ii) accessible genetic profile objects of the permissioned personal genetic profile of the genetic profile match.

For example, a permissioned personal genetic profile of a genetic profile match may include one or more accessible variant objects, each representing a particular variant of a particular SNP that the genetic profile match has. A subset of these accessible variant objects may match—that is, represent a same SNP variant and/or be associated with a same qualifier for the SNP variant as accessible variant objects in the permissioned personal genetic profile of the user. In this manner, a subset of matching SNPs for which both the user and the genetic profile match have a same variant and/or associated qualifier can be identified. Accordingly, graphics conveying information about each of the matching SNPs may be displayed in a visually associated manner with renderable data associated with a content object posted by the genetic profile match. In certain embodiments, for a particular matching SNP, the displayed graphics include, a representation of a gene with which the particular matching SNP is associated, such as a textual label identifying a name of the gene. In certain embodiments, the displayed graphics include graphics and/or text identifying the particular matching SNP, such as textual label corresponding to the SNP reference. In certain embodiments, if, for example, both the user and the genetic profile match have a same variant of the particular matching SNP, graphics and/or text corresponding to a measurement outcome associated with the variant may be displayed. In certain embodiments, for example if both the user and the genetic profile match have variants of the particular matching SNP that are associated with a same qualifier (e.g., the variants of the particular SNP that the user and the genetic profile match have may be different, but still associated with a same qualifier), graphics representing that qualifier may be displayed.

In certain embodiments, method 900 is used to allow a user to search for members of the social medial platform for genetic profile matches, a subset of which they may choose to “follow”, in order to be provided and/or notified of content postings by the followed members in their social media feed. For example, a user may access a member lookup view of the social media GUI to identify members of the social media platform to follow. The member lookup view may include graphical control elements such as selectable buttons and/or a lookup query dialog that allows a user to enter searchable text based on which member search results can be filtered.

In certain embodiments, the user may provide an input to identify genetic profile matches, for example by clicking on a particular selectable button (e.g., a “find genetic profile matches” button). Upon receipt of the user input to identify genetic profile matches, the steps 902, 904, 906, and 908 of method 900 are performed to identify genetic profile matches and present the user with a list of genetic profile matches. In particular, graphics and/or text corresponding to at least a portion of a biographical profile of the genetic profile matches may be caused to be rendered for presentation to the user. The rendered graphics and/or text may simply be a list of member identifiers (e.g., nicknames used by the members to identify themselves on the social media platform) and/or member names, or may also include additional information in the biographical profiles of the identified genetic profile matches.

The user input to identify genetic profile matches may also include a lookup query, at least a portion of which can be matched to data stored in biographical profiles of the social media platform members when identifying genetic profile matches. In this manner, the subset of social media platform members determined to be genetic profile matches are additionally filtered based on data in their biographical profiles. For example, the user lookup query may comprise one or more of (i) a member identifier [e.g., text that identifies a member (e.g., a nickname that the member uses to identify themselves on the social media platform that), (ii) a member name, and (iii) searchable text. The searchable text may include one or more keywords and/or keyphrases that can be matched to text, such as a short member description, in the biographical profile.

In certain embodiments, the user selects, from the list of genetic profile matches, one or more genetic profile matches as members to follow. For example, each of the genetic profile matches of the list may be displayed as a selectable icon, or in a visually associated manner with a selectable button. Upon receipt of a user selection of one or more of the genetic profile matches as members to follow, the selected genetic profile matches are added to a list of followed members. Content objects posted by such followed members are then displayed in the user's social media feed. In this manner, the systems and methods described herein allow for a user social media feed to be tailored based on genetic profile data in a variety of fashions.

### Computer System and Network Environment

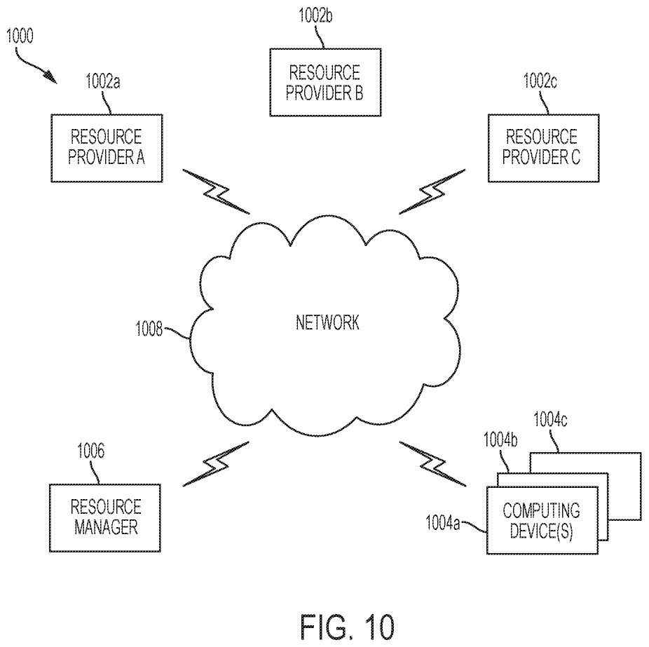

FIG. 10 shows an illustrative network environment 1000 for use in the methods and systems described herein. In brief overview, referring now to FIG. 10, a block diagram of an exemplary cloud computing environment 1000 is shown and described. The cloud computing environment 1000 may include one or more resource providers 1002a, 1002b, 1002c (collectively, 1002). Each resource provider 1002 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1002 may be connected to any other resource provider 1002 in the cloud computing environment 1000. In some implementations, the resource providers 1002 may be connected over a computer network 1008. Each resource provider 1002 may be connected to one or more computing device 1004a, 1004b, 1004c (collectively, 1004), over the computer network 1008.

The cloud computing environment 1000 may include a resource manager 1006. The resource manager 1006 may be connected to the resource providers 1002 and the computing devices 1004 over the computer network 1008. In some implementations, the resource manager 1006 may facilitate the provision of computing resources by one or more resource providers 1002 to one or more computing devices 1004. The resource manager 1006 may receive a request for a computing resource from a particular computing device 1004. The resource manager 1006 may identify one or more resource providers 1002 capable of providing the computing resource requested by the computing device 1004. The resource manager 1006 may select a resource provider 1002 to provide the computing resource. The resource manager 1006 may facilitate a connection between the resource provider 1002 and a particular computing device 1004. In some implementations, the resource manager 1006 may establish a connection between a particular resource provider 1002 and a particular computing device 1004. In some implementations, the resource manager 1006 may redirect a particular computing device 1004 to a particular resource provider 1002 with the requested computing resource.

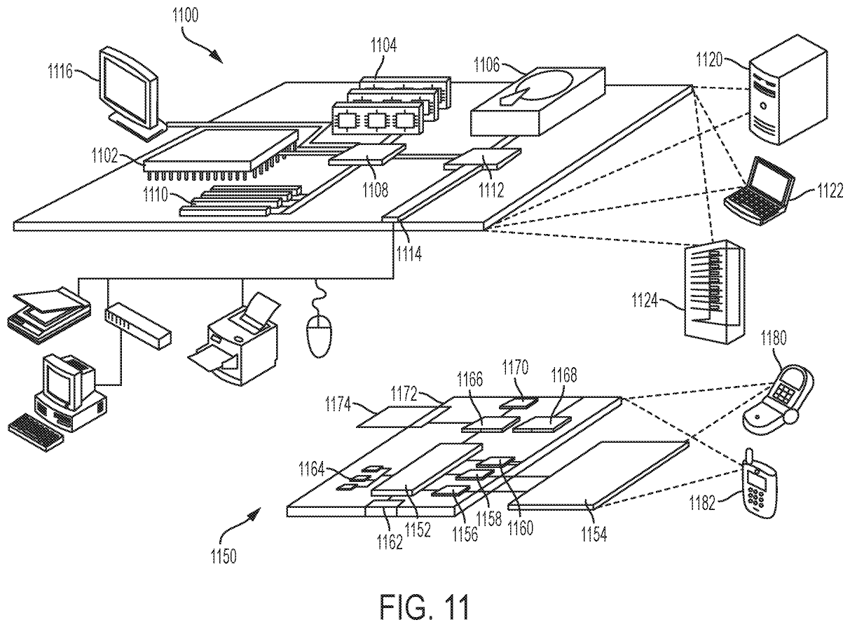

FIG. 11 shows an example of a computing device 1100 and a mobile computing device 1150 that can be used in the methods and systems described in this disclosure. The computing device 1100 is intended to represent various forms of digital computers, such as laptops, desktops, workstations, personal digital assistants, servers, blade servers, mainframes, and other appropriate computers. The mobile computing device 1150 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1100 includes a processor 1102, a memory 1104, a storage device 1106, a high-speed interface 1108 connecting to the memory 1104 and multiple high-speed expansion ports 1110, and a low-speed interface 1112 connecting to a low-speed expansion port 1114 and the storage device 1106. Each of the processor 1102, the memory 1104, the storage device 1106, the high-speed interface 1108, the high-speed expansion ports 1110, and the low-speed interface 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as a display 1116 coupled to the high-speed interface 1108. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1104 stores information within the computing device 1100. In some implementations, the memory 1104 is a volatile memory unit or units. In some implementations, the memory 1104 is a non-volatile memory unit or units. The memory 1104 may also be another form of computer-readable medium, such as a magnetic or optical disk.

The storage device 1106 is capable of providing mass storage for the computing device 1100. In some implementations, the storage device 1106 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1102), perform one or more methods, such as those described above. The instructions can also be stored by one or more storage devices such as computer- or machine-readable mediums (for example, the memory 1104, the storage device 1106, or memory on the processor 1102).

The high-speed interface 1108 manages bandwidth-intensive operations for the computing device 1100, while the low-speed interface 1112 manages lower bandwidth-intensive operations. Such allocation of functions is an example only. In some implementations, the high-speed interface 1108 is coupled to the memory 1104, the display 1116 (e.g., through a graphics processor or accelerator), and to the high-speed expansion ports 1110, which may accept various expansion cards (not shown). In the implementation, the low-speed interface 1112 is coupled to the storage device 1106 and the low-speed expansion port 1114. The low-speed expansion port 1114, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. In addition, it may be implemented in a personal computer such as a laptop computer 1122. It may also be implemented as part of a rack server system 1124. Alternatively, components from the computing device 1100 may be combined with other components in a mobile device (not shown), such as a mobile computing device 1150. Each of such devices may contain one or more of the computing device 1100 and the mobile computing device 1150, and an entire system may be made up of multiple computing devices communicating with each other.

The mobile computing device 1150 includes a processor 1152, a memory 1164, an input/output device such as a display 1154, a communication interface 1166, and a transceiver 1168, among other components. The mobile computing device 1150 may also be provided with a storage device, such as a micro-drive or other device, to provide additional storage. Each of the processor 1152, the memory 1164, the display 1154, the communication interface 1166, and the transceiver 1168, are interconnected using various buses, and several of the components may be mounted on a common motherboard or in other manners as appropriate.

The processor 1152 can execute instructions within the mobile computing device 1150, including instructions stored in the memory 1164. The processor 1152 may be implemented as a chipset of chips that include separate and multiple analog and digital processors. The processor 1152 may provide, for example, for coordination of the other components of the mobile computing device 1150, such as control of user interfaces, applications run by the mobile computing device 1150, and wireless communication by the mobile computing device 1150.

The processor 1152 may communicate with a user through a control interface 1158 and a display interface 1156 coupled to the display 1154. The display 1154 may be, for example, a TFT (Thin-Film-Transistor Liquid Crystal Display) display or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may provide communication with the processor 1152, so as to enable near area communication of the mobile computing device 1150 with other devices. The external interface 1162 may provide, for example, for wired communication in some implementations, or for wireless communication in other implementations, and multiple interfaces may also be used.

The memory 1164 stores information within the mobile computing device 1150. The memory 1164 can be implemented as one or more of a computer-readable medium or media, a volatile memory unit or units, or a non-volatile memory unit or units. An expansion memory 1174 may also be provided and connected to the mobile computing device 1150 through an expansion interface 1172, which may include, for example, a SIMM (Single In Line Memory Module) card interface. The expansion memory 1174 may provide extra storage space for the mobile computing device 1150, or may also store applications or other information for the mobile computing device 1150. Specifically, the expansion memory 1174 may include instructions to carry out or supplement the processes described above, and may include secure information also. Thus, for example, the expansion memory 1174 may be provided as a security module for the mobile computing device 1150, and may be programmed with instructions that permit secure use of the mobile computing device 1150. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1152), perform one or more methods, such as those described above. The instructions can also be stored by one or more storage devices, such as one or more computer- or machine-readable mediums (for example, the memory 1164, the expansion memory 1174, or memory on the processor 1152). In some implementations, the instructions can be received in a propagated signal, for example, over the transceiver 1168 or the external interface 1162.

The mobile computing device 1150 may communicate wirelessly through the communication interface 1166, which may include digital signal processing circuitry where necessary. The communication interface 1166 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1168 using a radio-frequency. In addition, short-range communication may occur, such as using a Bluetooth®, Wi-Fi™, or other such transceiver (not shown). In addition, a GPS (Global Positioning System) receiver module 1170 may provide additional navigation- and location-related wireless data to the mobile computing device 1150, which may be used as appropriate by applications running on the mobile computing device 1150.

The mobile computing device 1150 may also communicate audibly using an audio codec 1160, which may receive spoken information from a user and convert it to usable digital information. The audio codec 1160 may likewise generate audible sound for a user, such as through a speaker, e.g., in a handset of the mobile computing device 1150. Such sound may include sound from voice telephone calls, may include recorded sound (e.g., voice messages, music files, etc.) and may also include sound generated by applications operating on the mobile computing device 1150.

The mobile computing device 1150 may be implemented in a number of different forms, as shown in the figure. For example, it may be implemented as a cellular telephone 1180. It may also be implemented as part of a smart-phone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method of automatically identifying, presenting, and matching a user to members of a dating pool based on genetic interests and/or shared genetic information via a graphical user interface (GUI), the method comprising:
(a) accessing, by a processor of a computing device, a permissioned personal genetic profile of the user that identifies genetic interests of the user and/or genetic information to which the user has permitted access by the processor;
(b) accessing, by the processor, permissioned personal genetic profiles of each of one or more of the members of the dating pool;
(c) for each of a subset of the one or more members of the dating pool, determining, by the processor, the member to be a potential match based at least in part on the permissioned personal genetic profile of the user and the permissioned personal genetic profile of the member, thereby automatically identifying one or more potential matches for the user; and
(d) causing, by the processor, graphical rendering of, for presentation to the user within the GUI, graphics and/or text corresponding to at least a portion of a biographical profile of each of the one or more potential matches.

2. The method of claim 1, wherein at least one of the potential matches is linked to a first interest indicator corresponding to the user and having an affirmative value, and the method comprises:
(e) receiving, by the processor, via the GUI, a user input corresponding to second interest indicator corresponding to the at least one potential match and having an affirmative value;
(f) determining, by the processor, the at least one potential match to be a positive match for the user based on both the first and second interest indicators having affirmative values; and
(g) responsive to the determining the at least one potential match to be a positive match for the user, issuing, by the processor, a notification to the user of the positive match.

3. The method of any one of the preceding claims, wherein the permissioned personal genetic profile of the user comprises one or more genetic interest identifiers, each corresponding to a stored genomic object.

4. The method of any one of the preceding claims, wherein the permissioned personal genetic profile of the user comprises one or more accessible variant objects, each representing a particular variant of a particular SNP that the user has, and to which the user has granted access.

5. The method of any one of the preceding claims, wherein the permissioned personal genetic profile of the user comprises one or more secondary profile tags having been determined based on genotyping data for the user.

6. The method of any one of the preceding claims, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in a potential match with respect to the specific accessible genetic profile object of the user permissioned personal genetic profile.

7. The method of any one of the preceding claims, wherein, for each of one or more members of the dating pool, the permissioned personal genetic profile of the member comprises one or more genetic interest identifiers for the member, each corresponding to a stored genomic object.

8. The method of any one of the preceding claims, wherein, for each of one or more members of the dating pool, the permissioned personal genetic profile of the member comprises one or more accessible variant objects for the member, each representing a particular variant of a particular SNP that the member has, and to which the member has granted access.

9. The method of any one of the preceding claims, wherein, for each of one or more members of the dating pool, the permissioned personal genetic profile of the member comprises one or more secondary profile tags for the member, the secondary profile tags having been determined based on genotyping data for the member.

10. The method of any one of the preceding claims, wherein, for each of one or more members of the dating pool, the permissioned personal genetic profile of the member comprises one or more member-specified weighting values, each associated with a specific accessible genetic profile object of the permissioned personal genetic profile of the member and indicating a level of desired similarity and/or dissimilarity and/or compatibility in a potential match with respect to the specific accessible genetic profile object of the permissioned personal genetic profile of the member.

11. The method of any one of the preceding claims, wherein step (c) comprises, for each of one or more members of the dating pool:
computing a compatibility index for the member and the user based at least in part on (i) the permissioned personal genetic profile of the user and (ii) the permissioned personal genetic profile of the member; and
determining the member to be a potential match based at least in part on the computed compatibility index for the member and the user.

12. The method of any one of the preceding claims, wherein step (c) comprises determining the member to be a potential match based at least in part on at least a portion of a biographical profile of the member.

13. The method of any one of the preceding claims, the method comprising:
presenting, by the processor, a profile builder graphical user interface (GUI) for user selection and/or updating of permissioned personal genetic profile content;
receiving, by the processor, via the profile builder GUI, a user selection of one or more accessible genetic profile objects to include; and
storing, by the processor, within the permissioned personal genetic profile of the user, the user selection of the one or more accessible genetic profile objects to include.

14. The method of any one of the preceding claims, the method comprising:
accessing, by the processor, the permissioned personal genetic profile of the user;
presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receiving, by the processor, via the profile builder GUI, a user selection of one or more accessible genetic profile objects to exclude from their personal genetic profile; and
removing, by the processor, from the permissioned personal genetic profile of the user, the one or more accessible genetic profile objects selected by the user for exclusion from their permissioned personal genetic profile.

15. A method of automatically identifying and presenting to a user, one or more nearby genetic profile matches based on shared genetic interests and/or similar genotypes via a graphical user interface (GUI), the method comprising:
(a) identifying, by the processor, one or more nearby members of a genetic profile based matching service in geographic proximity to the user based on location data for the user and location data for each of the one or more members;
(b) accessing, by a processor of a computing device, a permissioned personal genetic profile of the user that identifies genetic interests of the user and/or genetic information to which the user has permitted access by the processor;
(c) accessing, by the processor, permissioned personal genetic profiles of each of one or more of the nearby members;
(d) for each of a subset of the one or more nearby members, determining, by the processor, the member to be a genetic profile match based on the permissioned personal genetic profile of the user and the permissioned personal genetic profile of the member, thereby automatically identifying one or more nearby genetic profile match(es) for the user; and
(e) causing, by the processor, graphical rendering of, for presentation to the user within the GUI graphics and/or text corresponding to at least a portion of a biographical profile of each of the one or more nearby genetic profile match(es).

16. The method of claim 15, wherein step (e) comprises causing graphical rendering of the graphics and/or text corresponding to the at least a portion of the biographical profile of each of the one or more nearby genetic profile match(es) in a location dependent fashion within the GUI based on the location data for each of the one or more nearby match(es) and the location data for the user.

17. The method of any one of claims 15-16, wherein the permissioned personal genetic profile of the user comprises one or more genetic interest identifiers, each corresponding to a stored genomic object.

18. The method of any one of claims 15-17, wherein the permissioned personal genetic profile of the user comprises one or more accessible variant objects, each representing a particular variant of a particular SNP that the user has, and to which the user has granted access.

19. The method of any one of claims 15-18, wherein the permissioned personal genetic profile of the user comprises one or more secondary profile tags having been determined based on genotyping data for the user.

20. The method of any one of claims 15-19, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in a potential match with respect to the specific accessible genetic profile object of the user permissioned personal genetic profile.

21. The method of any one of claims 15-20, wherein, for each of the one or more nearby members of the genetic profile based matching service, the permissioned personal genetic profile of the nearby member comprises one or more genetic interest identifiers for the nearby member, each corresponding to a stored genomic object.

22. The method of any one of claims 15-21, wherein, for each of the one or more nearby members of the genetic profile based matching service, the permissioned personal genetic profile of the nearby member comprises one or more accessible variant objects for the nearby member, each representing a particular variant of a particular SNP that the nearby member has, and to which the nearby member has granted access.

23. The method of any one of claims 15-22, wherein, for each of the one or more nearby members of the genetic profile based matching service, the permissioned personal genetic profile of the nearby member comprises one or more secondary profile tags for the nearby member, the secondary profile tags having been determined based on genotyping data for the nearby member.

24. The method of any one of claims 15-23, wherein, for each of the one or more nearby members of the genetic profile based matching service, the permissioned personal genetic profile of the nearby member comprises one or more member-specified weighting values, each associated with a specific accessible genetic profile object of the permissioned personal genetic profile of the nearby member and indicating a level of desired similarity and/or dissimilarity and/or compatibility in a genetic profile match with respect to the specific accessible genetic profile object of the permissioned personal genetic profile of the nearby member.

25. The method of any one of claims 15-24, wherein step (d) comprises, for each of one or more nearby members of the genetic profile based matching service:
computing a compatibility index for the nearby member and the user based at least in part on (i) the permissioned personal genetic profile of the user and (ii) the permissioned personal genetic profile of the nearby member; and
determining the nearby member to be a genetic profile match based at least in part on the computed compatibility index for the nearby member and the user.

26. The method of any one of claims 15-25, the method comprising:
presenting, by the processor, a profile builder graphical user interface (GUI) for user selection and/or updating of permissioned personal genetic profile content;
receiving, by the processor, via the profile builder GUI, a user selection of one or more accessible genetic profile objects to include; and
storing, by the processor, within the permissioned personal genetic profile of the user, the user selection of the one or more accessible genetic profile objects to include.

27. The method of any one of claims 15-26, the method comprising:
accessing, by the processor, the permissioned personal genetic profile of the user;
presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receiving, by the processor, via the profile builder GUI, a user selection of one or more accessible genetic profile objects to exclude from their personal genetic profile; and
removing, by the processor, from the permissioned personal genetic profile of the user, the one or more accessible genetic profile objects selected by the user for exclusion from their permissioned personal genetic profile.

28. A method of automatically identifying and presenting to a user, genetically tailored online content via a graphical user interface (GUI), the method comprising:
(a) accessing, by a processor of a computing device, a permissioned personal genetic profile of the user that identifies genetic interests of the user and/or genetic information to which the user has permitted access by the processor;
(b) accessing, by the processor, genetic profiles of each of one or more content objects;
(c) for each of a subset of the one or more content objects, determining, by the processor, the content object to be genetically relevant to the user based on the permissioned personal genetic profile of the user and the genetic profile of the content object, thereby automatically identifying one or more genetically relevant content objects for the user; and
(d) causing, by the processor, graphical rendering of, for presentation to the user within the GUI graphics and/or text corresponding to renderable data of the one or more genetically relevant content objects.

29. The method of claim 28, wherein the permissioned personal genetic profile of the user comprises one or more genetic interest identifiers, each corresponding to a stored genomic object.

30. The method of any one of claims 28-29, wherein the permissioned personal genetic profile of the user comprises one or more accessible variant objects, each representing a particular variant of a particular SNP that the user has, and to which the user has granted access.

31. The method of any one of claims 28-30, wherein the permissioned personal genetic profile of the user comprises one or more secondary profile tags having been determined based on genotyping data for the user.

32. The method of any one of claims 28-31, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in genetic profiles associated with content objects with respect to the specific accessible genetic profile object of the user permissioned personal genetic profile.

33. The method of any one of claims 28-32, wherein each of at least a portion of the one or more the content objects is a member post to an online forum, associated with a particular member and, for each member post, the genetic profile of the member post is a permissioned personal genetic profile of the particular member with which the post is associated.

34. The method of claim 33, wherein, for each member post, the permissioned personal genetic profile of the particular member with which the member post is associated comprises one or more genetic interest identifiers for the particular member, each corresponding to a stored genomic object.

35. The method of any one of claims 33-34, wherein, for each member post, the permissioned personal genetic profile of the particular member with which the member post is associated comprises one or more accessible variant objects for the particular member, each representing a particular variant of a particular SNP that the particular member has, and to which the particular member has granted access.

36. The method of any one of claims 33-35, wherein, for each member post, the permissioned personal genetic profile of the particular member with which the member post is associated comprises one or more secondary profile tags for the particular member, the secondary profile tags having been determined based on genotyping data for the particular member.

37. The method of any one of claims 33-36, wherein, for each member post, the permissioned personal genetic profile of the particular member with which the member post is associated comprises one or more member-specified weighting values, each associated with a specific accessible genetic profile object of the permissioned personal genetic profile of the particular member and indicating a level of similarity and/or dissimilarity and/or compatibility desired by the particular member in genetically relevant content that they view with respect to the specific accessible genetic profile object of the permissioned personal genetic profile of the particular member.

38. The method of any one of claims 33-37, wherein step (c) comprises, for each member post:
computing a compatibility index for the particular member with which the member post is associated and the user based at least in part on (i) the permissioned personal genetic profile of the user and (ii) the permissioned personal genetic profile of the particular member; and
determining the member post to be genetically relevant based at least in part on the computed compatibility index for the particular member and the user.

39. The method of any one of claims 28-38, wherein at least a portion of the one or more content objects are commercial posts, each having a content-specific genetic profile comprising one or more content-specific genetic profile objects that identify genetic interests for which the commercial post is relevant and/or genetic information that identifies a subset of genotypes for which the commercial post is relevant.

40. The method of claim 39, wherein, for each commercial post, the content-specific genetic profile associated with the commercial post comprises one or more genetic interest identifiers, each corresponding to a stored genomic object.

41. The method of any one of claims 39-40, wherein, for each commercial post, the content-specific genetic profile associated with the commercial post comprises one or more content-specific variant objects, each representing a particular variant of a particular SNP that identifies a subset of genotypes for which the commercial post is relevant.

42. The method of any one of claims 39-41, wherein, for each commercial post, the content-specific genetic profile of the commercial post comprises one or more content-specific secondary profile tags that are matchable to viable secondary profile tags of a permissioned personal genetic profile.

43. The method of any one of claims 39-42, wherein, for each commercial post, the content specific genetic profile of the commercial post comprises one or more content-specific weighting values, each associated with a particular content-specific genetic profile object of the content-specific genetic profile and indicating a level similarity and/or dissimilarity and/or compatibility of the commercial post with the user with respect to the particular content-specific genetic profile object of the content-specific genetic profile of the commercial post.

44. The method of any one of claims 39-43, wherein step (c) comprises, for each commercial post:
computing a compatibility index for the commercial post and the user based at least in part on (i) the permissioned personal genetic profile of the user and (ii) the content-specific genetic profile associated with the commercial post; and
determining the commercial post to be genetically relevant based at least in part on the computed compatibility index for the commercial post and the user.

45. The method of any one of claims 28-44, the method comprising:
presenting, by the processor, a profile builder graphical user interface (GUI) for user selection and/or updating of permissioned personal genetic profile content;
receiving, by the processor, via the profile builder GUI, a user selection of one or more accessible genetic profile objects to include; and
storing, by the processor, within the permissioned personal genetic profile of the user, the user selection of the one or more accessible genetic profile objects to include.

46. The method of any one of claims 28-45, the method comprising:
accessing, by the processor, the permissioned personal genetic profile of the user;
presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receiving, by the processor, via the profile builder GUI, a user selection of one or more accessible genetic profile objects to exclude from their personal genetic profile; and
removing, by the processor, from the permissioned personal genetic profile of the user, the one or more accessible genetic profile objects selected by the user for exclusion from their permissioned personal genetic profile.

47. A method of automatically managing and filtering social media interactions of a user with other social media platform members based on shared genetic interests and/or personal genetic data, the method comprising:
(a) accessing, by a processor of a computing device, a permissioned personal genetic profile of the user that identifies genetic interests of the user and/or genetic information that they have shared;
(b) accessing, by the processor, permissioned personal genetic profiles of each of one or more social media platform members;
(c) for each of a subset of the one or more social media platform members, determining, by the processor, the member to be a genetic profile match to the user based on the permissioned personal genetic profile of the user and the permissioned personal genetic profile of the social media platform member, thereby automatically identifying one or more genetic profile matches for the user; and
(d) causing, by the processor, graphical rendering of, for presentation to the user, within a graphical user interface (GUI), graphics and/or text corresponding to (i) at least a portion of a biographical profile of each of the one or more genetic profile matches for the user and/or (ii) set of renderable data associated with a content object associated each of at least a portion of the one or more genetic profile matches.

48. The method of claim 47, wherein the permissioned personal genetic profile of the user comprises one or more genetic interest identifiers, each corresponding to a stored genomic object.

49. The method of any one of claims 47-48, wherein the permissioned personal genetic profile of the user comprises one or more accessible variant objects, each representing a particular variant of a particular SNP that the user has, and to which the user has granted access.

50. The method of any one of claims 47-49, wherein the permissioned personal genetic profile of the user comprises one or more secondary profile tags having been determined based on genotyping data for the user.

51. The method of any one of claims 47-50, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility with members of the social media platform determined to be a genetic profile match with respect to the accessible genetic profile object of the user permissioned personal genetic profile.

52. The method of any one of claims 47-51, wherein, for each of the one or more social media platform members, the permissioned personal genetic profile of the social media platform member comprises one or more genetic interest identifiers for the social media platform member, each corresponding to a stored genomic object.

53. The method of any one of claims 47-52, wherein, for each of the one or more social media platform members, the permissioned personal genetic profile of the social media platform member comprises one or more accessible variant objects for the social media platform member, each representing a particular variant of a particular SNP that the social media platform member has, and to which the social media platform member has granted access.

54. The method of any one of claims 47-53, wherein, for each of the one or more social media platform members, the permissioned personal genetic profile of the social media platform member comprises one or more secondary profile tags for the social media platform member, the secondary profile tags having been determined based on genotyping data for the social media platform member.

55. The method of any one of claims 47-54, wherein, for each of the one or more social media platform members, the permissioned personal genetic profile of the social media platform member comprises one or more member-specified weighting values, each associated with a specific accessible genetic profile object of the permissioned personal genetic profile of the social media platform member and indicating a level of desired similarity and/or dissimilarity and/or compatibility in a genetic profile match with respect to the specific accessible genetic profile object of the permissioned personal genetic profile of the social media platform member.

56. The method of any one of claims 47-55, wherein step (c) comprises, for each of one or more social media platform members:
computing a compatibility index for the social media platform member and the user based at least in part on (i) the permissioned personal genetic profile of the user and (ii) the permissioned personal genetic profile of the social media platform member; and
determining the social media platform member to be a genetic profile match based at least in part on the computed compatibility index for the social media platform member and the user.

57. The method of any one of claims 47-56, the method comprising:
presenting, by the processor, a profile builder graphical user interface (GUI) for user selection and/or updating of permissioned personal genetic profile content;
receiving, by the processor, via the profile builder GUI, a user selection of one or more accessible genetic profile objects to include; and
storing, by the processor, within the permissioned personal genetic profile of the user, the user selection of the one or more accessible genetic profile objects to include.

58. The method of any one of claims 47-57, the method comprising:
accessing, by the processor, the permissioned personal genetic profile of the user;
presenting, by the processor, a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receiving, by the processor, via the profile builder GUI, a user selection of one or more accessible genetic profile objects to exclude from their personal genetic profile; and
removing, by the processor, from the permissioned personal genetic profile of the user, the one or more accessible genetic profile objects selected by the user for exclusion from their permissioned personal genetic profile.

59. The method of any one of claims 47 to 58, wherein step (d) comprises, for each set of renderable data associated with a content object, causing rendering of a graphics identifying one or more common genetic interests and/or traits between the user and the genetic profile match associated with the content object, each common genetic interest and/or trait corresponding to an accessible genetic profile object of the permissioned personal genetic profile of the genetic profile match that matches an accessible genetic profile object of the permissioned personal genetic profile of the user.

60. The method of claim 59, wherein the graphics identifying one or more common genetic interest and/or traits comprise, for each matching accessible genetic profile object, a graphical representation of a gene associated with both (i) the accessible genetic profile object of the permissioned personal genetic profile of the genetic profile match and (ii) the genetic profile object of the permissioned personal genetic profile of the user.

61. The method of any one of claims 47 to 60, the method comprising:
receiving, by the processor, a user input to identify genetic profile matches; and
performing steps (a)-(d) responsive to receipt of the user input to identify genetic profile matches.

62. The method of claim 61, wherein the user input to identify genetic profile matches comprises a lookup query, and wherein step (c) further comprises matching at least a portion of the lookup query to data stored in the biographical profile of each of the one or more genetic profile matches, such that the social media platform members are determined to be genetic profile matches of the user based further on the matching between the portion of the lookup query and the data stored in their biographical profiles.

63. The method of claim 62, wherein the lookup query comprises one or more of: (i) a member identifier, (ii) a member name, and (iii) searchable text.

64. The method of any one of claims 47 to 63, wherein step (d) comprises:
receiving, by the processor, a user input corresponding to a selection of at least a portion of the one or more genetic profile matches for the user for addition to a list of followed members;
adding, by the processor, the selected genetic profile matches to the list of followed members, thereby identifying the selected genetic profile matches as followed members; and
causing, by the processor, graphical rendering of, for presentation to the user, graphics and/or text corresponding to a set of renderable data associated with a content object associated with each of at least a portion of the followed members.

65. A system for automatically identifying, presenting, and matching a user to members of a dating pool based on genetic interests and/or shared genetic information via a graphical user interface (GUI), the system comprising:
a processor of a computing device; and
a memory having instructions stored thereon, wherein the instructions, when executed by the processor, cause the processor to:
(a) access a permissioned personal genetic profile of the user that identifies genetic interests of the user and/or genetic information to which the user has permitted access by the processor;
(b) access permissioned personal genetic profiles of each of one or more of the members of the dating pool;
(c) for each of a subset of the one or more members of the dating pool, determine the member to be a potential match based at least in part on the permissioned personal genetic profile of the user and the permissioned personal genetic profile of the member, thereby automatically identifying one or more potential matches for the user; and
(d) cause graphical rendering of, for presentation to the user within the GUI, graphics and/or text corresponding to at least a portion of a biographical profile of each of the one or more potential matches.

66. The system of claim 65, wherein at least one of the potential matches is linked to a first interest indicator corresponding to the user and having an affirmative value, and the instructions cause the processor to:
(e) receive, via the GUI, a user input corresponding to second interest indicator corresponding to the at least one potential match and having an affirmative value;
(f) determine, the at least one potential match to be a positive match for the user based on both the first and second interest indicators having affirmative values; and
(g) responsive to the determining the at least one potential match to be a positive match for the user, issue a notification to the user of the positive match.

67. The system of any one of claims 65 to 66, wherein the permissioned personal genetic profile of the user comprises one or more genetic interest identifiers, each corresponding to a stored genomic object.

68. The system of any one of claims 65 to 67, wherein the permissioned personal genetic profile of the user comprises one or more accessible variant objects, each representing a particular variant of a particular SNP that the user has, and to which the user has granted access.

69. The system of any one of claims 65 to 68, wherein the permissioned personal genetic profile of the user comprises one or more secondary profile tags having been determined based on genotyping data for the user.

70. The system of any one of claims 65 to 69, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in a potential match with respect to the specific accessible genetic profile object of the user permissioned personal genetic profile.

71. The system of any one of claims 69 to 70, wherein, for each of one or more members of the dating pool, the permissioned personal genetic profile of the member comprises one or more genetic interest identifiers for the member, each corresponding to a stored genomic object.

72. The system of any one of claims 65 to 71, wherein, for each of one or more members of the dating pool, the permissioned personal genetic profile of the member comprises one or more accessible variant objects for the member, each representing a particular variant of a particular SNP that the member has, and to which the member has granted access.

73. The system of any one of claims 65 to 72, wherein, for each of one or more members of the dating pool, the permissioned personal genetic profile of the member comprises one or more secondary profile tags for the member, the secondary profile tags having been determined based on genotyping data for the member.

74. The system of any one of claims 65 to 73, wherein, for each of one or more members of the dating pool, the permissioned personal genetic profile of the member comprises one or more member-specified weighting values, each associated with a specific accessible genetic profile object of the permissioned personal genetic profile of the member and indicating a level of desired similarity and/or dissimilarity and/or compatibility in a potential match with respect to the specific accessible genetic profile object of the permissioned personal genetic profile of the member.

75. The system of any one of claims 65 to 74, wherein at step (c), the instructions cause the processor to, for each of one or more members of the dating pool:
compute a compatibility index for the member and the user based at least in part on (i) the permissioned personal genetic profile of the user and (ii) the permissioned personal genetic profile of the member; and
determine the member to be a potential match based at least in part on the computed compatibility index for the member and the user.

76. The system of any one of claims 65 to 75, wherein at step (c), the instructions cause the processor to determine the member to be a potential match based at least in part on at least a portion of a biographical profile of the member.

77. The system of any one of claims 65 to 76, wherein the instructions cause the processor to:
present a profile builder graphical user interface (GUI) for user selection and/or updating of permissioned personal genetic profile content;
receive, via the profile builder GUI, a user selection of one or more accessible genetic profile objects to include; and
store within the permissioned personal genetic profile of the user, the user selection of the one or more accessible genetic profile objects to include.

78. The system of any one of claims 65 to 77, wherein the instructions cause the processor to:
access the permissioned personal genetic profile of the user;
present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receive, via the profile builder GUI, a user selection of one or more accessible genetic profile objects to exclude from their personal genetic profile; and
remove, from the permissioned personal genetic profile of the user, the one or more accessible genetic profile objects selected by the user for exclusion from their permissioned personal genetic profile.

79. A system for automatically identifying and presenting to a user, one or more nearby genetic profile matches based on shared genetic interests and/or similar (and/or compatible) genotypes via a graphical user interface (GUI), the system comprising:
a processor of a computing device; and
a memory having instructions stored thereon, wherein the instructions, when executed by the processor, cause the processor to:
(a) identify one or more nearby members of a genetic profile based matching service in geographic proximity to the user based on location for the user and location data for each of the one or more members;
(b) access a permissioned personal genetic profile of the user that identifies genetic interests of the user and/or genetic information to which the user has permitted access by the processor;
(c) access permissioned personal genetic profiles of each of one or more of the nearby members;
(d) for each of a subset of the one or more nearby members, determine the member to be a genetic profile match based on the permissioned personal genetic profile of the user and the permissioned personal genetic profile of the member, thereby automatically identifying one or more nearby genetic profile match(es) for the user; and
(e) cause graphical rendering of, for presentation to the user within the GUI graphics and/or text corresponding to at least a portion of a biographical profile of each of the one or more nearby genetic profile match(es).

80. The system of claim 79, wherein at step (e), the instructions cause the processor to cause graphical rendering of the graphics and/or text corresponding to the at least a portion of the biographical profile of each of the one or more nearby genetic profile match(es) in a location dependent fashion within the GUI based on the location data for each of the one or more nearby match(es) and the location data for the user.

81. The system of any one of claims 79-80, wherein the permissioned personal genetic profile of the user comprises one or more genetic interest identifiers, each corresponding to a stored genomic object.

82. The system of any one of claims 80-81, wherein the permissioned personal genetic profile of the user comprises one or more accessible variant objects, each representing a particular variant of a particular SNP that the user has, and to which the user has granted access.

83. The system of any one of claims 79-82, wherein the permissioned personal genetic profile of the user comprises one or more secondary profile tags having been determined based on genotyping data for the user.

84. The system of any one of claims 79-83, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in a potential match with respect to the specific accessible genetic profile object of the user permissioned personal genetic profile.

85. The system of any one of claims 79-84, wherein, for each of the one or more nearby members of the genetic profile based matching service, the permissioned personal genetic profile of the nearby member comprises one or more genetic interest identifiers for the nearby member, each corresponding to a stored genomic object.

86. The system of any one of claims 79-85, wherein, for each of the one or more nearby members of the genetic profile based matching service, the permissioned personal genetic profile of the nearby member comprises one or more accessible variant objects for the nearby member, each representing a particular variant of a particular SNP that the nearby member has, and to which the nearby member has granted access.

87. The system of any one of claims 79-86, wherein, for each of the one or more nearby members of the genetic profile based matching service, the permissioned personal genetic profile of the nearby member comprises one or more secondary profile tags for the nearby member, the secondary profile tags having been determined based on genotyping data for the nearby member.

88. The system of any one of claims 79-87, wherein, for each of the one or more nearby members of the genetic profile based matching service, the permissioned personal genetic profile of the nearby member comprises one or more member-specified weighting values, each associated with a specific accessible genetic profile object of the permissioned personal genetic profile of the nearby member and indicating a level of desired similarity and/or dissimilarity and/or compatibility in a genetic profile match with respect to the specific accessible genetic profile object of the permissioned personal genetic profile of the nearby member.

89. The system of any one of claims 79-88, wherein at step (d), the instructions cause the processor to, for each of one or more nearby members of the genetic profile based matching service:
compute a compatibility index for the nearby member and the user based at least in part on (i) the permissioned personal genetic profile of the user and (ii) the permissioned personal genetic profile of the nearby member; and
determine the nearby member to be a genetic profile match based at least in part on the computed compatibility index for the nearby member and the user.

90. The system of any one of claims 79-89, wherein the instructions cause the processor to:
present a profile builder graphical user interface (GUI) for user selection and/or updating of permissioned personal genetic profile content;
receive, via the profile builder GUI, a user selection of one or more accessible genetic profile objects to include; and
storing, by the processor, within the permissioned personal genetic profile of the user, the user selection of the one or more accessible genetic profile objects to include.

91. The system of any one of claims 79-90, wherein the instructions cause the processor to:
access the permissioned personal genetic profile of the user;
present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receive, via the profile builder GUI, a user selection of one or more accessible genetic profile objects to exclude from their personal genetic profile; and
remove, from the permissioned personal genetic profile of the user, the one or more accessible genetic profile objects selected by the user for exclusion from their permissioned personal genetic profile.

92. A system for automatically identifying and presenting to a user, genetically tailored online content via a graphical user interface (GUI), the system comprising:
a processor of a computing device; and
a memory having instructions stored thereon, wherein the instructions, when executed by the processor, cause the processor to:
(a) access a permissioned personal genetic profile of the user that identifies genetic interests of the user and/or genetic information to which the user has permitted access by the processor;
(b) access genetic profiles of each of one or more content objects;
(c) for each of a subset of the one or more content objects, determine the content object to be genetically relevant to the user based on the permissioned personal genetic profile of the user and the genetic profile of the content object, thereby automatically identifying one or more genetically relevant content objects for the user; and
(d) cause graphical rendering of, for presentation to the user within the GUI graphics and/or text corresponding to renderable data of the one or more genetically relevant content objects.

93. The system of claim 92, wherein the permissioned personal genetic profile of the user comprises one or more genetic interest identifiers, each corresponding to a stored genomic object.

94. The system of any one of claims 92-93, wherein the permissioned personal genetic profile of the user comprises one or more accessible variant objects, each representing a particular variant of a particular SNP that the user has, and to which the user has granted access.

95. The system of any one of claims 92-94, wherein the permissioned personal genetic profile of the user comprises one or more secondary profile tags having been determined based on genotyping data for the user.

96. The system of any one of claims 92-95, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility in genetic profiles associated with content objects with respect to the specific accessible genetic profile object of the user permissioned personal genetic profile.

97. The system of any one of claims 92-96, wherein each of at least a portion of the one or more the content objects is a member post to an online forum, associated with a particular member and, for each member post, the genetic profile of the member post is a permissioned personal genetic profile of the particular member with which the post is associated.

98. The system of claim 97, wherein, for each member post, the permissioned personal genetic profile of the particular member with which the member post is associated comprises one or more genetic interest identifiers for the particular member, each corresponding to a stored genomic object.

99. The system of any one of claims 97-98, wherein, for each member post, the permissioned personal genetic profile of the particular member with which the member post is associated comprises one or more accessible variant objects for the particular member, each representing a particular variant of a particular SNP that the particular member has, and to which the particular member has granted access.

100. The system of any one of claims 97-99, wherein, for each member post, the permissioned personal genetic profile of the particular member with which the member post is associated comprises one or more secondary profile tags for the particular member, the secondary profile tags having been determined based on genotyping data for the particular member.

101. The system of any one of claims 97-100, wherein, for each member post, the permissioned personal genetic profile of the particular member with which the member post is associated comprises one or more member-specified weighting values, each associated with a specific accessible genetic profile object of the permissioned personal genetic profile of the particular member and indicating a level of similarity and/or dissimilarity and/or compatibility desired by the particular member in genetically relevant content that they view with respect to the specific accessible genetic profile object of the permissioned personal genetic profile of the particular member.

102. The system of any one of claims 97-101, wherein at step (c) the instructions cause the processor to, for each member post:
compute a compatibility index for the particular member with which the member post is associated and the user based at least in part on (i) the permissioned personal genetic profile of the user and (ii) the permissioned personal genetic profile of the particular member; and
determine the member post to be genetically relevant based at least in part on the computed compatibility index for the particular member and the user.

103. The system of any one of claims 92-102, wherein at least a portion of the one or more content objects are commercial posts, each having a content-specific genetic profile comprising one or more content-specific genetic profile objects that identify genetic interests for which the commercial post is relevant and/or genetic information that identifies a subset of genotypes for which the commercial post is relevant.

104. The system of claim 103, wherein, for each commercial post, the content-specific genetic profile associated with the commercial post comprises one or more genetic interest identifiers, each corresponding to a stored genomic object.

105. The system of any one of claims 103-104, wherein, for each commercial post, the content-specific genetic profile associated with the commercial post comprises one or more content-specific variant objects, each representing a particular variant of a particular SNP that identifies a subset of genotypes for which the commercial post is relevant.

106. The system of any one of claims 103-105, wherein, for each commercial post, the content-specific genetic profile of the commercial post comprises one or more content-specific secondary profile tags that are matchable to viable secondary profile tags of a permissioned personal genetic profile.

107. The system of any one of claims 103-106, wherein, for each commercial post, the content specific genetic profile of the commercial post comprises one or more content-specific weighting values, each associated with a particular content-specific genetic profile object of the content-specific genetic profile and indicating a level similarity and/or dissimilarity and/or compatibility of the commercial post with the user with respect to the particular content-specific genetic profile object of the content-specific genetic profile of the commercial post.

108. The system of any one of claims 104-107, wherein at step (c), the instructions cause the processor to, for each commercial post:
compute a compatibility index for the commercial post and the user based at least in part on (i) the permissioned personal genetic profile of the user and (ii) the content-specific genetic profile associated with the commercial post; and
determine the commercial post to be genetically relevant based at least in part on the computed compatibility index for the commercial post and the user.

109. The system of any one of claims 92-108, wherein the instructions cause the processor to:
present a profile builder graphical user interface (GUI) for user selection and/or updating of permissioned personal genetic profile content;
receive via the profile builder GUI, a user selection of one or more accessible genetic profile objects to include; and
store, within the permissioned personal genetic profile of the user, the user selection of the one or more accessible genetic profile objects to include.

110. The system of any one of claims 92-109, wherein the instructions cause the processor to:
access the permissioned personal genetic profile of the user;
present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receive, via the profile builder GUI, a user selection of one or more accessible genetic profile objects to exclude from their personal genetic profile; and
remove, from the permissioned personal genetic profile of the user, the one or more accessible genetic profile objects selected by the user for exclusion from their permissioned personal genetic profile.

111. A system for automatically managing and filtering social media interactions of a user with other social media platform members based on shared genetic interests and/or personal genetic data, the system comprising:
a processor of a computing device; and
a memory having instructions stored thereon, wherein the instructions, when executed by the processor, cause the processor to:
(a) access a permissioned personal genetic profile of the user that identifies genetic interests of the user and/or genetic information that they have shared;
(b) access permissioned personal genetic profiles of each of one or more social media platform members;
(c) for each of a subset of the one or more social media platform members, determine the member to be a genetic profile match to the user based on the permissioned personal genetic profile of the user and the permissioned personal genetic profile of the social media platform member, thereby automatically identifying one or more genetic profile matches for the user; and
(d) cause graphical rendering of, for presentation to the user, within a graphical user interface (GUI), graphics and/or text corresponding to (i) at least a portion of a biographical profile of each of the one or more genetic profile matches for the user and/or (ii) set of renderable data associated with a content object associated each of at least a portion of the one or more genetic profile matches.

112. The system of claim 111, wherein the permissioned personal genetic profile of the user comprises one or more genetic interest identifiers, each corresponding to a stored genomic object.

113. The system of any one of claims 111-112, wherein the permissioned personal genetic profile of the user comprises one or more accessible variant objects, each representing a particular variant of a particular SNP that the user has, and to which the user has granted access.

114. The system of any one of claims 111-113, wherein the permissioned personal genetic profile of the user comprises one or more secondary profile tags having been determined based on genotyping data for the user.

115. The system of any one of claims 111-114, wherein the permissioned personal genetic profile of the user comprises one or more user-specified weighting values, each associated with a specific accessible genetic profile object of the user permissioned personal genetic profile and indicating a level of desired similarity and/or dissimilarity and/or compatibility with members of the social media platform determined to be a genetic profile match with respect to the accessible genetic profile object of the user permissioned personal genetic profile.

116. The system of any one of claims 111-115, wherein, for each of the one or more social media platform members, the permissioned personal genetic profile of the social media platform member comprises one or more genetic interest identifiers for the social media platform member, each corresponding to a stored genomic object.

117. The system of any one of claims 111-116, wherein, for each of the one or more social media platform members, the permissioned personal genetic profile of the social media platform member comprises one or more accessible variant objects for the social media platform member, each representing a particular variant of a particular SNP that the social media platform member has, and to which the social media platform member has granted access.

118. The system of any one of claims 111-117, wherein, for each of the one or more social media platform members, the permissioned personal genetic profile of the social media platform member comprises one or more secondary profile tags for the social media platform member, the secondary profile tags having been determined based on genotyping data for the social media platform member.

119. The system of any one of claims 111-118, wherein, for each of the one or more social media platform members, the permissioned personal genetic profile of the social media platform member comprises one or more member-specified weighting values, each associated with a specific accessible genetic profile object of the permissioned personal genetic profile of the social media platform member and indicating a level of desired similarity and/or dissimilarity and/or compatibility in a genetic profile match with respect to the specific accessible genetic profile object of the permissioned personal genetic profile of the social media platform member.

120. The system of any one of claims 111-119, wherein at step (c), the instructions cause the processor to, for each of one or more social media platform members:
compute a compatibility index for the social media platform member and the user based at least in part on (i) the permissioned personal genetic profile of the user and (ii) the permissioned personal genetic profile of the social media platform member; and
determine the social media platform member to be a genetic profile match based at least in part on the computed compatibility index for the social media platform member and the user.

121. The system of any one of claims 111-120, wherein the instructions cause the processor to:
present a profile builder graphical user interface (GUI) for user selection and/or updating of permissioned personal genetic profile content;
receive, via the profile builder GUI, a user selection of one or more accessible genetic profile objects to include; and
store, within the permissioned personal genetic profile of the user, the user selection of the one or more accessible genetic profile objects to include.

122. The method of any one of claims 111-121, wherein the instructions cause the processor to:
access the permissioned personal genetic profile of the user;
present a profile builder graphical user interface (GUI) for user selection and/or updating of permissioned personal genetic profile content, the profile builder GUI comprising, for each of one or more accessible genetic profile objects stored in the permissioned personal genetic profile of the user, selectable graphics and/or text identifying the accessible genetic profile object;
receive, via the profile builder GUI, a user selection of one or more accessible genetic profile objects to exclude from their personal genetic profile; and
remove, from the permissioned personal genetic profile of the user, the one or more accessible genetic profile objects selected by the user for exclusion from their permissioned personal genetic profile.

123. The system of any one of claims 111 to 122, wherein at step (d), the instructions cause the processor to, for each set of renderable data associated with a content object, causing rendering of a graphics identifying one or more common genetic interests and/or traits between the user and the genetic profile match associated with the content object, each common genetic interest and/or trait corresponding to an accessible genetic profile object of the permissioned personal genetic profile of the genetic profile match that matches an accessible genetic profile object of the permissioned personal genetic profile of the user.

124. The system of claim 123, wherein the graphics identifying one or more common genetic interest and/or traits comprise, for each matching accessible genetic profile object, a graphical representation of a gene associated with both (i) the accessible genetic profile object of the permissioned personal genetic profile of the genetic profile match and (ii) the genetic profile object of the permissioned personal genetic profile of the user.

125. The system of any one of claims 111 to 124, wherein the instructions cause the processor to:
receive a user input to identify genetic profile matches; and
responsive to receipt of the user input to identify genetic profile matches, perform steps (a)-(d).

126. The system of claim 125, wherein the user input to identify genetic profile matches comprises a lookup query, and wherein step (c) further comprises matching at least a portion of the lookup query to data stored in the biographical profile of each of the one or more genetic profile matches, such that the social media platform members are determined to be genetic profile matches of the user based further on the matching between the portion of the lookup query and the data stored in their biographical profiles.

127. The system of claim 126, wherein the lookup query comprises one or more of: (i) a member identifier, (ii) a member name, and (iii) searchable text.

128. The system of any one of claims 111 to 127, wherein at step (d) the instructions cause the processor to:
receive a user input corresponding to a selection of at least a portion of the one or more genetic profile matches for the user for addition to a list of followed members;
add the selected genetic profile matches to the list of followed members, thereby identifying the selected genetic profile matches as followed members; and
cause graphical rendering of, for presentation to the user, graphics and/or text corresponding to a set of renderable data associated with a content object associated with each of at least a portion of the followed members.
